(19) United States
(12) Patent Application Publication
(10) Pub. No.: US 2025/0177142 A1
(43) Pub. Date: Jun. 5, 2025

(54) Coaptation of the Mitral Valve Using a Tether That Runs from within the Right Atrium to an Outer Wall of the Left Ventricle

(71) Applicant: Ventricord Ltd., Zikhron Ya'akov (IL)

(72) Inventor: David ALON, Zichron Yaacov (IL)

(21) Appl. No.: 18/971,832

(22) Filed: Dec. 6, 2024

Related U.S. Application Data
(63) Continuation-in-part of application No. PCT/IB2024/051104, filed on Feb. 6, 2024.
(60) Provisional application No. 63/527,440, filed on Jul. 18, 2023.
(60) Provisional application No. 63/443,751, filed on Feb. 7, 2023.

(51) Int. Cl. A61F 2/24; A61B 17/04; A61B 17/34

(52) U.S. Cl. CPC A61F 2/2487; A61B 17/3468; A61B 2017/0409; A61B 2017/0464; A61F 2220/0075

(57) ABSTRACT

Mitral valve regurgitation can be ameliorated by creating one passageway between the right atrium and the left ventricle at a portion of the tricuspid annulus that is directly above the interventricular septum and creating another passageway through an outer wall of the left ventricle near at least one of the left-ventricular papillary muscles. A first support member is positioned within the right atrium in contact with a septal portion of the tricuspid annulus, and a support plate is positioned against the outer wall of the left ventricle adjacent to the second passageway. The support plate is pulled towards the first support member using a tether under tension that runs between the first support plate and the first support member and passes through both passageways. And the tension in the tether moves the roots of the chordae closer to the mitral valve, which ameliorates the mitral valve regurgitation.

### CROSS REFERENCE TO RELATED APPLICATIONS

This Application is a continuation-in-part of international application PCT/IB2024/051104 (filed Feb. 6, 2024), which claims the benefit of U.S. Provisional Applications 63/443,751 (filed Feb. 7, 2023) and 63/527,440 (filed Jul. 18, 2023), each of which is incorporated herein by reference in its entirety.

### FIELD OF THE INVENTION

The present invention relates to medical devices, in particular devices for repairing insufficiency (leaking) of the Mitral valve.

### BACKGROUND

Ischemic mitral regurgitation (IMR) is mitral regurgitation (MR) caused by chronic changes of left ventricle (LV) structure and function due to ischemic heart disease. IMR represents the valvular consequences of increased tethering forces that are applied to the mitral valve by the chordae tendinea, and these increased tethering forces reduce the sealing properties of the coaptation surfaces in the mitral valve's leaflets. The restricted motion of the valve leaflets can often eliminate coaptation altogether, resulting in severe leaking of the valve.

The Coapsys device was a notable attempt to reduce IMR by reducing the tethering forces that the chordae exert on the leaflets of the mitral valve. The Coapsys device used two extracardiac pads that were surgically implanted and connected by a flexible, trans-ventricular sub-valvular cord. By shortening that cord after the pads were in place, the ventricular walls were drawn together, which reduced the pulling force that the chordae exerted on the leaflets. This, in turn, improved coaptation of the valve leaflets and reduced the MR grade.

But the Coapsys device had two significant shortcomings. First, installing the device required a highly invasive median sternotomy to obtain full access to the outside of the subject's heart. And second, a significantly-sized puncture of the heart was needed to install the device, which gave rise to an associated risk of bleeding.

### SUMMARY OF THE INVENTION

One aspect of the invention is directed to a first method of ameliorating mitral regurgitation in a heart within a subject's body. The first method comprises positioning a first needle within a left ventricle of the heart, with a first tether affixed to the first needle; and pushing the first needle through a posterior wall of the left ventricle at a first location until a distal portion of the first tether passes through the posterior wall and exits the heart. The first method also comprises advancing the first tether in a distal direction until a first portion of the first tether exits the subject's body; and affixing a first expandable support structure to the first portion of the first tether. The first method also comprises retracting the first tether and moving the first expandable support structure in a proximal direction until the first expandable support structure reaches an outer surface of the posterior wall adjacent to the first location; and expanding the first expandable support structure so that a region of contact between the first expandable support structure and the outer surface of the posterior wall is formed. And the first method also comprises securing the first tether to a portion of the subject's body that is located anteriorly with respect to the left ventricle.

In some instances of the first method, the first location is between the papillary muscles of the heart. In some instances of the first method, the first expandable support structure comprises a first polymer bag, the expanding comprises injecting a liquid material into the first polymer bag, and the liquid material is configured to subsequently solidify. In some instances of the first method, the first expandable support structure comprises a plurality of metal arms, and the expanding comprises moving the metal arms from an initial collapsed position to a final expanded position.

In some instances of the first method, the region of contact between the first expandable support structure and the outer surface of the posterior wall has an area of at least 2 cm². In some instances of the first method, the region of contact between the first expandable support structure and the outer surface of the posterior wall has an area of at least 5 cm². In some instances of the first method, the region of contact between the first expandable support structure and the outer surface of the posterior wall has an area between 6 and 15 cm².

In some instances of the first method, the securing of the first tether comprises positioning a second needle within the left ventricle of the heart, with a second tether affixed to the second needle; pushing the second needle through a septum of the heart at a second location and through an outer wall of the subject's right ventricle until a distal portion of the second tether passes through the outer wall of the right ventricle and exits the heart; advancing the second tether in a distal direction until a first portion of the second tether exits the subject's body; attaching the second tether to the first tether so as to form a joined tether; moving a second expandable support structure in a proximal direction over the joined tether until the second expandable support structure enters the right ventricle and reaches the septum adjacent to the second location; expanding the second expandable support structure so that a region of contact between the second expandable support structure and the septum is formed; and attaching the second expandable support structure to the joined tether in a manner that establishes tension in the first tether. The tension pulls the first expandable support structure towards the second expandable support structure.

Optionally, in the instances described in the previous paragraph, the region of contact between the first expandable support structure and the outer surface of the posterior wall has an area of at least 5 cm², and the region of contact between the second expandable support structure and the septum has an area of at least 5 cm².

In some instances of the first method, the securing of the first tether comprises positioning a second needle within the left ventricle of the heart, with a second tether affixed to the second needle; pushing the second needle through a septum of the heart and through an outer wall of the subject's right ventricle at a second location until a distal portion of the second tether passes through the outer wall of the right ventricle and exits the heart; advancing the second tether in a distal direction until a first portion of the second tether exits the subject's body; attaching the second tether to the first tether so as to form a joined tether; moving a second expandable support structure in a proximal direction over the joined tether until the second expandable support structure reaches an outer surface of the right ventricle adjacent to the second location; expanding the second expandable support structure so that a region of contact between the second expandable support structure and the outer surface of the right ventricle is formed; and attaching the second expandable support structure to the joined tether in a manner that establishes tension in the first tether. The tension pulls the first expandable support structure towards the second expandable support structure.

Optionally, in the instances described in the previous paragraph, the region of contact between the first expandable support structure and the outer surface of the posterior wall has an area of at least 5 cm², and the region of contact between the second expandable support structure and the outer surface of the right ventricle has an area of at least 5 cm².

In some instances of the first method, the securing of the first tether comprises positioning a second needle within the left ventricle of the heart, with a second tether affixed to the second needle; pushing the second needle through a septum of the heart and through an outer wall of the subject's right ventricle until a distal portion of the second tether passes through the outer wall of the right ventricle and exits the heart; affixing the second tether to a bone in the subject's body; and attaching the second tether to the first tether in a manner that establishes tension in the first tether. The tension pulls the first expandable support structure towards the bone.

In some instances of the first method, the securing of the first tether comprises positioning a second needle within the left ventricle of the heart, with a second tether affixed to the second needle; pushing the second needle through an anterior wall of the left ventricle at a second location until a distal portion of the second tether exits the heart; advancing the second tether in a distal direction until a first portion of the second tether exits the subject's body; attaching the second tether to the first tether so as to form a joined tether; moving a second expandable support structure in a proximal direction over the joined tether until the second expandable support structure reaches an outer surface of the anterior wall adjacent to the second location; expanding the second expandable support structure so that a region of contact between the second expandable support structure and the outer surface of the anterior wall is formed; and attaching the second expandable support structure to the joined tether in a manner that establishes tension in the first tether. The tension pulls the first expandable support structure towards the second expandable support structure.

In some instances of the first method, the securing of the first tether comprises positioning a second needle within the left ventricle of the heart, with a second tether affixed to the second needle; pushing the second needle through an anterior wall of the left ventricle and a posterior wall of the subject's right atrium at a second location located just above the subject's tricuspid anulus, and subsequently pushing the second needle through an outer wall of the right atrium until a distal portion of the second tether passes through the outer wall of the right atrium and exits the heart; advancing the second tether in a distal direction until a first portion of the second tether exits the subject's body; attaching the second tether to the first tether so as to form a joined tether; moving a second expandable support structure in a proximal direction over the joined tether until the second expandable support structure reaches, within the right atrium, the posterior wall of the right atrium, just above the subject's tricuspid annulus, adjacent to the second location; expanding the second expandable support structure so that a region of contact between the second expandable support structure and a surface of the tricuspid annulus is formed; and attaching the second expandable support structure to the joined tether in a manner that establishes tension in the first tether. The tension pulls the first expandable support structure towards the second expandable support structure.

Another aspect of the invention is directed to a first apparatus for ameliorating mitral regurgitation in a heart within a subject's body. The first apparatus comprises a first pad, a second pad, and a tether. The first pad is positioned against an outer surface of a posterior wall of the heart, at a location that is within 3 cm of a midpoint between the centers of the two papillary muscles. The second pad is positioned within the heart's right atrium, against a surface of the heart's tricuspid annulus that is adjacent to a posterior wall of the right atrium. And the tether runs between the first pad and the second pad, and is under tension so that the first pad is pulled towards the second pad.

In some embodiments of the first apparatus, the first pad is positioned against the outer surface of the posterior wall of the heart, at a location that is within 2 cm of the midpoint between the centers of the two papillary muscles. In some embodiments of the first apparatus, the first pad is formed by injecting a first liquid material into a first polymer bag, and allowing the first liquid material to solidify, and the second pad is formed by injecting a second liquid material into a second polymer bag, and allowing the second liquid material to solidify.

In some embodiments of the first apparatus, a region of contact between the first pad and the outer surface of the posterior wall has an area of at least 2 cm². In some embodiments of the first apparatus, a region of contact between the first pad and the outer surface of the posterior wall has an area of at least 5 cm². In some embodiments of the first apparatus, a region of contact between the first pad and the outer surface of the posterior wall has an area between 6 and 15 cm².

Another aspect of the invention is directed to a second method of ameliorating mitral regurgitation in a heart within a subject's body. The second method comprises creating a first passageway between the right atrium and the left ventricle at a portion of the tricuspid annulus that is directly above the interventricular septum; creating a second passageway through an outer wall of the left ventricle near at least one of the left-ventricular papillary muscles; positioning a support bar within the right atrium in contact with a septal portion of the tricuspid annulus; positioning a first support plate against the outer wall of the left ventricle adjacent to the second passageway; and pulling the first support plate towards the support bar using a first tether under tension. The first tether runs between the first support plate and the support bar and passes through both the first passageway and the second passageway, and a first end of the first tether is secured to the first support plate.

In some instances of the second method, the second passageway is located between the two left-ventricular papillary muscles. In some instances of the second method, a second end of the first tether is secured to the support bar.

In some instances of the second method, the first support plate has an area of 3-13 cm², and the support bar has a length of 10-20 mm and a width of 3-8 mm. In some instances of the second method, the first support plate has an area of 4-16 cm², and the support bar has a length of 15-25 mm and a width of 3-8 mm. In some instances of the second method, the first support plate has an area of 7-20 cm², and the support bar has a length of 20-30 mm and a width of 3-8 mm.

In some instances of the second method, the positioning of the first support plate against the outer wall of the left ventricle comprises: positioning a polymer bag outside the outer wall of the left ventricle adjacent to the second passageway; injecting a liquid material into the polymer bag; and allowing the liquid material to solidify. The solidification of the liquid material forms the first support plate.

Some instances of the second method further comprise creating a third passageway through an outer wall of the right ventricle near at least one right-ventricular papillary muscle; positioning a second support plate against an outer wall of the right ventricle adjacent to the third passageway; and pulling the second support plate towards the support bar using a second tether under tension. In these instances, the second tether runs between the second support plate and the support bar, and a first end of the second tether is secured to the second support plate.

Some instances of the second method further comprise positioning a second support member against an outer wall of the heart at a second location adjacent to an anterior leaflet of the tricuspid valve; and pulling the second support member towards the support bar using a second tether under tension. In these instances, the second tether runs between the second support member and the support bar, and a first end of the second tether is secured to the second support member.

Some instances of the second method further comprise positioning a second support member against an outer wall of the heart at a second location adjacent to a posterior leaflet of a mitral valve; and pulling the second support member towards the support bar using a second tether under tension. In these instances, the second tether runs between the second support member and the support bar, and a first end of the second tether is secured to the second support member.

Another aspect of the invention is directed to a third method of ameliorating mitral regurgitation in a heart within a subject's body. The third method comprises (a) introducing a first needle into the right atrium, with a first tether affixed to the first needle; (b) pushing the first needle through the cardiac skeleton into the left ventricle at a location that is directly above the interventricular septum to create a first passageway between the right atrium and the left ventricle; (c) pushing the first needle through an outer wall of the left ventricle at a location between the left-ventricular papillary muscles to create a second passageway; (d) advancing the first needle in a distal direction until a distal portion of the first tether exits the heart; (e) advancing the first tether in a distal direction until a portion of the first tether exits the subject's body; (f) affixing a first support plate to the first tether; and (g) retracting the first tether and moving the first support plate in a proximal direction between the subject's ribs until the first support plate reaches an outer surface of the outer wall of the left ventricle adjacent to the second passageway. The third method also comprises positioning a support bar within the right atrium adjacent to the first passageway. The third method also comprises, after steps (a) through (g), pulling the first tether in a proximal direction while the support bar is positioned adjacent to the first passageway, and subsequently securing the first tether to the support bar under tension.

Some instances of the third method further comprise introducing a second needle into the right ventricle, with a second tether affixed to the second needle; pushing the second needle through an outer wall of the right ventricle at a location between the subject's right-ventricular papillary muscles to create a third passageway; advancing the second needle in a distal direction until a distal portion of the second tether exits the heart; advancing the second tether in a distal direction until a portion of the second tether exits the subject's body; advancing a second support plate over the second tether in a proximal direction until the second support plate reaches an outer surface of the outer wall of the right ventricle adjacent to the third passageway; pushing the second support plate against the outer wall of the right ventricle; and securing one portion of the second tether to the second support plate and securing another portion of the second tether to the support bar so that the second tether is under tension.

Some instances of the third method further comprise introducing a second needle into the right ventricle, with a second tether affixed to the second needle; pushing the second needle through an outer wall of the right ventricle at a location between the subject's right-ventricular papillary muscles to create a third passageway; advancing the second needle in a distal direction until a distal portion of the second tether exits the heart; advancing the second tether in a distal direction until a portion of the second tether exits the subject's body; affixing a second support plate to the second tether; retracting the second tether and moving the second support plate in a proximal direction between the subject's ribs until the second support plate reaches an outer surface of the outer wall of the right ventricle adjacent to the third passageway; and pulling the second tether in a proximal direction, and subsequently securing the second tether to the support bar under tension.

Some instances of the third method further comprise introducing a second needle into the right atrium, with a second tether affixed to the second needle; pushing the second needle through an anterior outer wall of the right atrium at a location above the tricuspid valve to create a third passageway; advancing the second needle in a distal direction until a distal portion of the second tether exits the heart; advancing the second tether in a distal direction until a portion of the second tether exits the subject's body; advancing a curved support member over the second tether in a proximal direction until the curved support member reaches an outer surface of the outer wall of the right atrium adjacent to the third passageway, wherein the curved support member has a curvature that fits the outer wall of the right atrium; pushing the curved support member against the outer wall of the right atrium; and securing one portion of the second tether to the curved support member and securing another portion of the second tether to the support bar so that the second tether is under tension.

Some instances of the third method further comprise introducing a second needle into the right atrium, with a second tether affixed to the second needle; pushing the second needle through an anterior outer wall of the right atrium at a location above the tricuspid valve to create a third passageway; advancing the second needle in a distal direction until a distal portion of the second tether exits the heart; advancing the second tether in a distal direction until a portion of the second tether exits the subject's body; affixing a curved support member to the second tether, wherein the curved support member has a curvature that fits the outer wall of the right atrium; retracting the second tether and moving the curved support member in a proximal direction between the subject's ribs until the curved support member reaches an outer surface of the outer wall of the right atrium adjacent to the third passageway; and pulling the second tether in a proximal direction, and subsequently securing the second tether to the support bar under tension.

Some instances of the third method further comprise introducing a second needle into the right atrium, with a second tether affixed to the second needle; advancing the second needle through the heart until the second needle enters the left atrium; pushing the second needle through a posterior outer wall of the left atrium at a location above the mitral valve to create a third passageway; advancing the second needle in a distal direction until a distal portion of the second tether exits the heart; advancing the second tether in a distal direction until a portion of the second tether exits the subject's body; advancing a curved support member over the second tether in a proximal direction until the curved support member reaches an outer surface of the outer wall of the left atrium adjacent to the third passageway, wherein the curved support member has a curvature that fits the outer wall of the left atrium; pushing the curved support member against the outer wall of the left atrium; and securing one portion of the second tether to the curved support member and securing another portion of the second tether to the support bar so that the second tether is under tension.

Some instances of the third method further comprise introducing a second needle into the right atrium, with a second tether affixed to the second needle; advancing the second needle through the heart until the second needle enters the left atrium; pushing the second needle through a posterior outer wall of the left atrium at a location above the mitral valve to create a third passageway; advancing the second needle in a distal direction until a distal portion of the second tether exits the heart; advancing the second tether in a distal direction until a portion of the second tether exits the subject's body; affixing a curved support member to the second tether, wherein the curved support member has a curvature that fits the outer wall of the left atrium; retracting the second tether and moving the curved support member in a proximal direction between the subject's ribs until the curved support member reaches an outer surface of the outer wall of the left atrium adjacent to the third passageway; and pulling the second tether in a proximal direction, and subsequently securing the second tether to the support bar under tension.

Another aspect of the invention is directed to a second apparatus for ameliorating mitral regurgitation in a heart within a subject's body. The second apparatus comprises a support bar, a first support plate, and a first tether. The support bar is positioned within the subject's right atrium, adjacent to a first passageway that runs between the subject's right atrium and the subject's left ventricle. The first passageway is located directly above the subject's interventricular septum. The first support plate is positioned against an outer surface of a subject's left ventricle, adjacent to a second passageway that runs through an outer wall of a subject's left ventricle. And the second passageway is located between the subject's left-ventricular papillary muscles. The first tether runs between the support bar and the first support plate and passes through both the first passageway and the second passageway. A first portion of the first tether is secured to the support bar and a second portion of the first tether is secured to the first support plate. And the first tether is under tension so that the first support plate is pulled towards the support bar.

In some embodiments of the second apparatus, the first support plate has an area of 3-13 cm², the support bar has a length of 10-20 mm and a width of 3-8 mm, and the first tether has a length of 60-85 mm. In some embodiments of the second apparatus, the first support plate has an area of 4-16 cm², the support bar has a length of 15-25 mm and a width of 3-8 mm, and the first tether has a length of 65-90 mm. In some embodiments of the second apparatus, the first support plate has an area of 7-20 cm², the support bar has a length of 20-30 mm and a width of 3-8 mm, and the first tether has a length of 70-95 mm.

Some embodiments of the second apparatus further comprise a second support plate and a second tether. The second support plate is positioned against an outer surface of an outer wall of the subject's right ventricle, adjacent to a third passageway that runs through the outer wall of the subject's right ventricle, and the second passageway is located between the subject's right-ventricular papillary muscles. The second tether runs between the support bar and the second support plate and passes through the third passageway. A first portion of the second tether is secured to the support bar and a second portion of the second tether is secured to the second support plate. And the second tether is under tension so that the second support plate is pulled towards the support bar.

Some embodiments of the second apparatus further comprise a curved support member and a second tether. The curved support member is positioned against an outer surface of an outer wall of the subject's right atrium, adjacent to a third passageway that runs through the outer wall of the subject's right atrium, and the third passageway is located above the subject's tricuspid valve. The second tether runs between the support bar and the curved support member and passes through the third passageway. A first portion of the second tether is secured to the support bar and a second portion of the second tether is secured to the curved support member. And the second tether is under tension so that the curved support member is pulled towards the support bar.

Some embodiments of the second apparatus further comprise a curved support member and a second tether. The curved support member is positioned against an outer surface of an outer wall of the subject's left atrium, adjacent to a third passageway that runs through the outer wall of the subject's left atrium, and the third passageway is located above the subject's mitral valve. The second tether runs between the support bar and the curved support member and passes through the third passageway. A first portion of the second tether is secured to the support bar and a second portion of the second tether is secured to the curved support member. And the second tether is under tension so that the curved support member is pulled towards the support bar.

Another aspect of the invention is directed to a fourth method of ameliorating mitral regurgitation in a heart within a subject's body. The heart has a right atrium, a left atrium, a right ventricle, a left ventricle, two left-ventricular papillary muscles, an interventricular septum, a tricuspid valve, a mitral valve, and a cardiac skeleton. The fourth method comprises (a) introducing a first needle into the right atrium, with a first advancing tether affixed to the first needle; (b) creating a first passageway between the right atrium and the left ventricle through the cardiac skeleton at a location that is directly above the interventricular septum and pushing the first needle through the first passageway into the left ventricle; (c) creating a second passageway through an outer wall of the left ventricle at a location between the left-ventricular papillary muscles and pushing the first needle through the second passageway; (d) advancing the first needle in a distal direction until a distal portion of the first advancing tether exits the heart; (e) advancing the first advancing tether in a distal direction until a portion of the first advancing tether exits the subject's body; (f) securing a first support plate to a first retracting tether that is threaded through both the first passageway and the second passageway; and (g) retracting the first retracting tether and moving the first support plate in a proximal direction between the subject's ribs until the first support plate reaches an outer surface of the outer wall of the left ventricle adjacent to the second passageway. The fourth method also comprises positioning a support bar within the right atrium adjacent to the first passageway. And the fourth method also comprises, after steps (a) through (g), pulling the first retracting tether in a proximal direction while the support bar is positioned adjacent to the first passageway, and subsequently securing the first retracting tether to the support bar under tension.

In some instances of the fourth method, a single tether serves as both the first advancing tether and the first retracting tether. In some instances of the fourth method, the first advancing tether and the first retracting tether are distinct from each other, and the first advancing tether is used to thread the first retracting tether through both the first passageway and the second passageway.

Some instances of the fourth method further comprise introducing a second needle into the right ventricle, with a second tether affixed to the second needle; creating a third passageway through an outer wall of the right ventricle at a location between the subject's right-ventricular papillary muscles and pushing the second needle through the third passageway; advancing the second needle in a distal direction until a distal portion of the second tether exits the heart; advancing the second tether in a distal direction until a portion of the second tether exits the subject's body; advancing a second support plate over the second tether in a proximal direction until the second support plate reaches an outer surface of the outer wall of the right ventricle adjacent to the third passageway; pushing the second support plate against the outer wall of the right ventricle; and securing one portion of the second tether to the second support plate and securing another portion of the second tether to the support bar so that the second tether is under tension.

Some instances of the fourth method further comprise introducing a second needle into the right ventricle, with a second advancing tether affixed to the second needle; creating a third passageway through an outer wall of the right ventricle at a location between the subject's right-ventricular papillary muscles and pushing the second needle through the third passageway; advancing the second needle in a distal direction until a distal portion of the second advancing tether exits the heart; advancing the second advancing tether in a distal direction until a portion of the second advancing tether exits the subject's body; securing a second support plate to a second retracting tether that is threaded through the third passageway; retracting the second retracting tether and moving the second support plate in a proximal direction between the subject's ribs until the second support plate reaches an outer surface of the outer wall of the right ventricle adjacent to the third passageway; and pulling the second retracting tether in a proximal direction, and subsequently securing the second retracting tether to the support bar under tension.

Some instances of the fourth method further comprise introducing a second needle into the right atrium, with a second tether affixed to the second needle; creating a third passageway through an anterior outer wall of the right atrium at a location above the tricuspid valve and pushing the second needle through the third passageway; advancing the second needle in a distal direction until a distal portion of the second tether exits the heart; advancing the second tether in a distal direction until a portion of the second tether exits the subject's body; advancing a curved support member over the second tether in a proximal direction until the curved support member reaches an outer surface of the outer wall of the right atrium adjacent to the third passageway, wherein the curved support member has a curvature that fits the outer wall of the right atrium; pushing the curved support member against the outer wall of the right atrium; and securing one portion of the second tether to the curved support member and securing another portion of the second tether to the support bar so that the second tether is under tension.

Some instances of the fourth method further comprise introducing a second needle into the right atrium, with a second advancing tether affixed to the second needle; creating a third passageway through an anterior outer wall of the right atrium at a location above the tricuspid valve and pushing the second needle through the third passageway; advancing the second needle in a distal direction until a distal portion of the second advancing tether exits the heart; advancing the second advancing tether in a distal direction until a portion of the second advancing tether exits the subject's body; securing a curved support member to a second retracting tether that is threaded through the third passageway, wherein the curved support member has a curvature that fits the outer wall of the right atrium; retracting the second retracting tether and moving the curved support member in a proximal direction between the subject's ribs until the curved support member reaches an outer surface of the outer wall of the right atrium adjacent to the third passageway; and pulling the second retracting tether in a proximal direction, and subsequently securing the second tether to the support bar under tension.

Some instances of the fourth method further comprise introducing a second needle into the right atrium, with a second tether affixed to the second needle; advancing the second needle through the heart until the second needle enters the left atrium; creating a third passageway through a posterior outer wall of the left atrium at a location above the mitral valve and pushing the second needle through the third passageway; advancing the second needle in a distal direction until a distal portion of the second tether exits the heart; advancing the second tether in a distal direction until a portion of the second tether exits the subject's body; advancing a curved support member over the second tether in a proximal direction until the curved support member reaches an outer surface of the outer wall of the left atrium adjacent to the third passageway, wherein the curved support member has a curvature that fits the outer wall of the left atrium; pushing the curved support member against the outer wall of the left atrium; and securing one portion of the second tether to the curved support member and securing another portion of the second tether to the support bar so that the second tether is under tension.

Some instances of the fourth method further comprise introducing a second needle into the right atrium, with a second advancing tether affixed to the second needle; advancing the second needle through the heart until the second needle enters the left atrium; creating a third passageway through a posterior outer wall of the left atrium at a location above the mitral valve and pushing the second needle through the third passageway; advancing the second needle in a distal direction until a distal portion of the second advancing tether exits the heart; advancing the second advancing tether in a distal direction until a portion of the second advancing tether exits the subject's body; securing a curved support member to a second retracting tether that is threaded through the third passageway, wherein the curved support member has a curvature that fits the outer wall of the left atrium; retracting the second retracting tether and moving the curved support member in a proximal direction between the subject's ribs until the curved support member reaches an outer surface of the outer wall of the left atrium adjacent to the third passageway; and pulling the second retracting tether in a proximal direction, and subsequently securing the second tether to the support bar under tension.

Another aspect of the invention is directed to a third apparatus for ameliorating mitral regurgitation in a heart within a subject's body. The heart has two left-ventricular papillary muscles, each of which has a respective center. The third apparatus comprises a support plate, a support bar, and a tether. The support plate is positioned against an outer surface of a posterior wall of the heart, at a location that is within 3 cm of a midpoint between the centers of the two left-ventricular papillary muscles. The support bar is positioned within the heart's right atrium, against a surface of the heart's tricuspid annulus that is adjacent to a posterior wall of the right atrium. And the tether runs between the support plate and the support bar. The tether is under tension so that the support plate is pulled towards the support bar.

In some embodiments of the third apparatus, the support plate is positioned against the outer surface of the posterior wall of the heart, at a location that is within 2 cm of the midpoint between the centers of the two left-ventricular papillary muscles.

In some embodiments of the third apparatus, the support plate is formed by injecting a first liquid material into a first polymer bag, and allowing the first liquid material to solidify. Optionally, in these embodiments, the support bar can be formed by injecting a second liquid material into a second polymer bag, and allowing the second liquid material to solidify.

In some embodiments of the third apparatus, a region of contact between the support plate and the outer surface of the posterior wall has an area of at least 2 cm². In some embodiments of the third apparatus, a region of contact between the support plate and the outer surface of the posterior wall has an area of at least 5 cm². In some embodiments of the third apparatus, a region of contact between the support plate and the outer surface of the posterior wall has an area between 6 and 15 cm².

Another aspect of the invention is directed to a fifth method of ameliorating mitral regurgitation in a heart within a subject's body. The heart has a right atrium, a right ventricle, a left ventricle, two left-ventricular papillary muscles, an interventricular septum, a tricuspid valve, and a tricuspid annulus. The fifth method comprises creating a first passageway between the right atrium and the left ventricle at a septal portion of the tricuspid annulus that is directly above the interventricular septum; creating a second passageway through an outer wall of the left ventricle near at least one of the left-ventricular papillary muscles; positioning a first support member within the right atrium in contact with a septal portion of the tricuspid annulus; positioning a first support plate against the outer wall of the left ventricle adjacent to the second passageway; and pulling the first support plate towards the first support member using a first tether under tension. The first tether runs between the first support plate and the first support member and passes through both the first passageway and the second passageway. And a first end of the first tether is secured to the first support plate.

In some instances of the fifth method, the second passageway is located between the two left-ventricular papillary muscles. In some instances of the fifth method, a second end of the first tether is secured to the first support member.

In some instances of the fifth method, the first support member is non-elongated. Optionally, in these instances, the first support plate has an area of 3-13 cm², the first support member has a length of 3-8 mm, and the first tether has a length of 60-85 mm.

In some instances of the fifth method, the first support member is non-elongated, the first support plate has an area of 4-16 cm, the first support member has a length of 7-12 mm, and the first tether has a length of 65-90 mm.

In some instances of the fifth method, the first support member is non-elongated, the first support plate has an area of 7-20 cm², the first support member has a length of 11-16 mm, and the first tether has a length of 70-95 mm.

In some instances of the fifth method, the first support member is bar-shaped. Optionally, in these instances, the first support plate has an area of 3-13 cm², and the first support member has a length of 10-20 mm and a width of 3-8 mm.

In some instances of the fifth method, the first support member is bar-shaped, the first support plate has an area of 4-16 cm², and the first support member has a length of 15-25 mm and a width of 3-8 mm.

In some instances of the fifth method, the first support member is bar-shaped, the first support plate has an area of 7-20 cm², and the first support member has a length of 20-30 mm and a width of 3-8 mm.

In some instances of the fifth method, the positioning of the first support plate against the outer wall of the left ventricle comprises positioning a polymer bag outside the outer wall of the left ventricle adjacent to the second passageway; injecting a liquid material into the polymer bag; and allowing the liquid material to solidify. The solidification of the liquid material forms the first support plate.

Some instances of the fifth method further comprise creating a third passageway through an outer wall of the right ventricle near at least one right-ventricular papillary muscle; positioning a second support plate against an outer wall of the right ventricle adjacent to the third passageway; and pulling the second support plate towards the first support member using a second tether under tension. The second tether runs between the second support plate and the first support member, and a first end of the second tether is secured to the second support plate.

Some instances of the fifth method further comprise positioning a second support member against an outer wall of the heart at a second location adjacent to an anterior leaflet of the tricuspid valve; and pulling the second support member towards the first support member using a second tether under tension. The second tether runs between the second support member and the first support member, and a first end of the second tether is secured to the second support member.

Some instances of the fifth method further comprise positioning a second support member against an outer wall of the heart at a second location adjacent to a posterior leaflet of a mitral valve; and pulling the second support member towards the first support member using a second tether under tension. The second tether runs between the second support member and the first support member, and a first end of the second tether is secured to the second support member.

Another aspect of the invention is directed to a sixth method of ameliorating mitral regurgitation in a heart within a subject's body. The heart has a right atrium, a left atrium, a right ventricle, a left ventricle, two left-ventricular papillary muscles, an interventricular septum, a tricuspid valve, a mitral valve, and a cardiac skeleton. The sixth method comprises (a) introducing a first needle into the right atrium, with a first advancing tether affixed to the first needle; (b) creating a first passageway between the right atrium and the left ventricle through the cardiac skeleton at a location that is directly above the interventricular septum and pushing the first needle through the first passageway into the left ventricle; (c) creating a second passageway through an outer wall of the left ventricle at a location between the left-ventricular papillary muscles and pushing the first needle through the second passageway; (d) advancing the first needle in a distal direction until a distal portion of the first advancing tether exits the heart; (e) advancing the first advancing tether in a distal direction until a portion of the first advancing tether exits the subject's body; (f) securing a first support plate to a first retracting tether that is threaded through both the first passageway and the second passageway; and (g) retracting the first retracting tether and moving the first support plate in a proximal direction between the subject's ribs until the first support plate reaches an outer surface of the outer wall of the left ventricle adjacent to the second passageway. The sixth method also comprises positioning a first support member within the right atrium adjacent to the first passageway. The sixth method also comprises, after steps (a) through (g), pulling the first retracting tether in a proximal direction while the first support member is positioned adjacent to the first passageway, and subsequently securing the first retracting tether to the first support member under tension.

In some instances of the sixth method, a single tether serves as both the first advancing tether and the first retracting tether. In some instances of the sixth method, the first advancing tether and the first retracting tether are distinct from each other, and the first advancing tether is used to thread the first retracting tether through both the first passageway and the second passageway.

In some instances of the sixth method, the first support member is non-elongated. In some instances of the sixth method, the first support member is bar-shaped.

Some instances of the sixth method further comprise introducing a second needle into the right ventricle, with a second tether affixed to the second needle; creating a third passageway through an outer wall of the right ventricle at a location between the subject's right-ventricular papillary muscles and pushing the second needle through the third passageway; advancing the second needle in a distal direction until a distal portion of the second tether exits the heart; advancing the second tether in a distal direction until a portion of the second tether exits the subject's body; advancing a second support plate over the second tether in a proximal direction until the second support plate reaches an outer surface of the outer wall of the right ventricle adjacent to the third passageway; pushing the second support plate against the outer wall of the right ventricle; and securing one portion of the second tether to the second support plate, and securing another portion of the second tether to the first support member so that the second tether is under tension.

Some instances of the sixth method further comprise introducing a second needle into the right ventricle, with a second advancing tether affixed to the second needle; creating a third passageway through an outer wall of the right ventricle at a location between the subject's right-ventricular papillary muscles and pushing the second needle through the third passageway; advancing the second needle in a distal direction until a distal portion of the second advancing tether exits the heart; advancing the second advancing tether in a distal direction until a portion of the second advancing tether exits the subject's body; securing a second support plate to a second retracting tether that is threaded through the third passageway; retracting the second retracting tether and moving the second support plate in a proximal direction between the subject's ribs until the second support plate reaches an outer surface of the outer wall of the right ventricle adjacent to the third passageway; and pulling the second retracting tether in a proximal direction, and subsequently securing the second retracting tether to the first support member under tension.

Some instances of the sixth method further comprise introducing a second needle into the right atrium, with a second tether affixed to the second needle; creating a third passageway through an anterior outer wall of the right atrium at a location above the tricuspid valve and pushing the second needle through the third passageway; advancing the second needle in a distal direction until a distal portion of the second tether exits the heart; advancing the second tether in a distal direction until a portion of the second tether exits the subject's body; advancing a curved support member over the second tether in a proximal direction until the curved support member reaches an outer surface of the outer wall of the right atrium adjacent to the third passageway, wherein the curved support member has a curvature that fits the outer wall of the right atrium; pushing the curved support member against the outer wall of the right atrium; and securing one portion of the second tether to the curved support member, and securing another portion of the second tether to the first support member so that the second tether is under tension.

Some instances of the sixth method further comprise introducing a second needle into the right atrium, with a second advancing tether affixed to the second needle; creating a third passageway through an anterior outer wall of the right atrium at a location above the tricuspid valve and pushing the second needle through the third passageway; advancing the second needle in a distal direction until a distal portion of the second advancing tether exits the heart; advancing the second advancing tether in a distal direction until a portion of the second advancing tether exits the subject's body; securing a curved support member to a second retracting tether that is threaded through the third passageway, wherein the curved support member has a curvature that fits the outer wall of the right atrium; retracting the second retracting tether and moving the curved support member in a proximal direction between the subject's ribs until the curved support member reaches an outer surface of the outer wall of the right atrium adjacent to the third passageway; and pulling the second retracting tether in a proximal direction, and subsequently securing the second tether to the first support member under tension.

Some instances of the sixth method further comprise introducing a second needle into the right atrium, with a second tether affixed to the second needle; advancing the second needle through the heart until the second needle enters the left atrium; creating a third passageway through a posterior outer wall of the left atrium at a location above the mitral valve and pushing the second needle through the third passageway; advancing the second needle in a distal direction until a distal portion of the second tether exits the heart; advancing the second tether in a distal direction until a portion of the second tether exits the subject's body; advancing a curved support member over the second tether in a proximal direction until the curved support member reaches an outer surface of the outer wall of the left atrium adjacent to the third passageway, wherein the curved support member has a curvature that fits the outer wall of the left atrium; pushing the curved support member against the outer wall of the left atrium; and securing one portion of the second tether to the curved support member and securing another portion of the second tether to the first support member so that the second tether is under tension.

Some instances of the sixth method further comprise introducing a second needle into the right atrium, with a second advancing tether affixed to the second needle; advancing the second needle through the heart until the second needle enters the left atrium; creating a third passageway through a posterior outer wall of the left atrium at a location above the mitral valve and pushing the second needle through the third passageway; advancing the second needle in a distal direction until a distal portion of the second advancing tether exits the heart; advancing the second advancing tether in a distal direction until a portion of the second advancing tether exits the subject's body; securing a curved support member to a second retracting tether that is threaded through the third passageway, wherein the curved support member has a curvature that fits the outer wall of the left atrium; retracting the second retracting tether and moving the curved support member in a proximal direction between the subject's ribs until the curved support member reaches an outer surface of the outer wall of the left atrium adjacent to the third passageway; and pulling the second retracting tether in a proximal direction, and subsequently securing the second tether to the first support member under tension.

Another aspect of the invention is directed to a fourth apparatus for ameliorating mitral regurgitation in a heart within a subject's body. The fourth apparatus comprises a first support member, a first support plate, and a first tether. The first support member is positioned within the subject's right atrium, adjacent to a first passageway that runs between the subject's right atrium and the subject's left ventricle. The first passageway is located directly above the subject's interventricular septum. The first support plate is positioned against an outer surface of a subject's left ventricle, adjacent to a second passageway that runs through an outer wall of a subject's left ventricle. The second passageway is located between the subject's left-ventricular papillary muscles. The first tether runs between the first support member and the first support plate and passes through both the first passageway and the second passageway. A first portion of the first tether is secured to the first support member and a second portion of the first tether is secured to the first support plate. And the first tether is under tension so that the first support plate is pulled towards the first support member.

In some embodiments of the fourth apparatus, the first support plate has an area of 3-13 cm², the first support member is a support bar having a length of 10-20 mm and a width of 3-8 mm, and the first tether has a length of 60-85 mm.

In some embodiments of the fourth apparatus, the first support plate has an area of 4-16 cm², the first support member is a support bar having a length of 15-25 mm and a width of 3-8 mm, and the first tether has a length of 65-90 mm.

In some embodiments of the fourth apparatus, the first support plate has an area of 7-20 cm², the first support member is a support bar having a length of 20-30 mm and a width of 3-8 mm, and the first tether has a length of 70-95 mm.

In some embodiments of the fourth apparatus, the first support plate has an area of 3-13 cm², the first support member is non-elongated and has a length of 3-8 mm, and the first tether has a length of 60-85 mm.

In some embodiments of the fourth apparatus, the first support plate has an area of 4-16 cm², the first support member is non-elongated and has a length of 7-12 mm, and the first tether has a length of 65-90 mm.

In some embodiments of the fourth apparatus, the first support plate has an area of 7-20 cm², the first support member is non-elongated and has a length of 11-16 mm, and the first tether has a length of 70-95 mm.

Some embodiments of the fourth apparatus further comprise a second support plate and a second tether. The second support plate is positioned against an outer surface of an outer wall of the subject's right ventricle, adjacent to a third passageway that runs through the outer wall of the subject's right ventricle. The second passageway is located between the subject's right-ventricular papillary muscles. The second tether runs between the first support member and the second support plate and passes through the third passageway. A first portion of the second tether is secured to the first support member and a second portion of the second tether is secured to the second support plate. And the second tether is under tension so that the second support plate is pulled towards the first support member.

Some embodiments of the fourth apparatus further comprise a curved support member and a second tether. The curved support member is positioned against an outer surface of an outer wall of the subject's right atrium, adjacent to a third passageway that runs through the outer wall of the subject's right atrium. The third passageway is located above the subject's tricuspid valve. The second tether runs between the first support member and the curved support member and passes through the third passageway. A first portion of the second tether is secured to the first support member and a second portion of the second tether is secured to the curved support member. And the second tether is under tension so that the curved support member is pulled towards the first support member.

Some embodiments of the fourth apparatus further comprise a curved support member and a second tether. The curved support member is positioned against an outer surface of an outer wall of the subject's left atrium, adjacent to a third passageway that runs through the outer wall of the subject's left atrium. The third passageway is located above the subject's mitral valve. The second tether runs between the first support member and the curved support member and passes through the third passageway. A first portion of the second tether is secured to the first support member and a second portion of the second tether is secured to the curved support member. And the second tether is under tension so that the curved support member is pulled towards the first support member.

Another aspect of the invention is directed to a fifth apparatus for ameliorating mitral regurgitation in a heart within a subject's body. The heart has two left-ventricular papillary muscles, each of which has a respective center. The fifth apparatus comprises a support plate, a first support member, and a tether. The support plate is configured for being positioned against an outer surface of a posterior wall of the heart, at a location that is within 3 cm of a midpoint between the centers of the two left-ventricular papillary muscles. The first support member is configured for being positioned within the heart's right atrium, against a surface of the heart's tricuspid annulus that is adjacent to a posterior wall of the right atrium. And the tether runs between the support plate and the first support member. The tether is under tension so that the support plate is pulled towards the first support member.

In some embodiments of the fifth apparatus, the support plate is configured to be positioned against the outer surface of the posterior wall of the heart, at a location that is within 2 cm of the midpoint between the centers of the two left-ventricular papillary muscles.

In some embodiments of the fifth apparatus, the first support member is configured to be positioned adjacent to a first passageway that runs between the subject's right atrium and the subject's left ventricle, the support plate is configured to be positioned adjacent to a second passageway that runs through an outer wall of a subject's left ventricle, the tether is configured to pass through both the first passageway and the second passageway, and a first portion of the tether is secured to the first support member and a second portion of the tether is secured to the support plate.

In some embodiments of the fifth apparatus, the first support member is non-elongated. Optionally, in these embodiments, the support plate has an area of 3-13 cm², the first support member has a length of 3-8 mm, and the tether has a length of 60-85 mm.

In some embodiments of the fifth apparatus, the first support member is non-elongated, the support plate has an area of 4-16 cm, the first support member has a length of 7-12 mm, and the tether has a length of 65-90 mm.

In some embodiments of the fifth apparatus, the first support member is non-elongated, the support plate has an area of 7-20 cm², the first support member has a length of 11-16 mm, and the tether has a length of 70-95 mm.

In some embodiments of the fifth apparatus, the first support member is bar-shaped. Optionally, in these embodiments, the support plate has an area of 3-13 cm², the first support member has a length of 10-20 mm and a width of 3-8 mm, and the tether has a length of 60-85 mm.

In some embodiments of the fifth apparatus, the first support member is bar-shaped, the support plate has an area of 4-16 cm², the first support member has a length of 15-25 mm and a width of 3-8 mm, and the tether has a length of 65-90 mm.

In some embodiments of the fifth apparatus, the first support member is bar-shaped, the support plate has an area of 7-20 cm², the first support member has a length of 20-30 mm and a width of 3-8 mm, and the tether has a length of 70-95 mm.

### BRIEF DESCRIPTION OF THE DRAWINGS

FIG. 1A depicts the heart of a healthy subject.

FIG. 1B depicts the heart of a subject who suffers from IMR.

FIG. 2 depicts a step of a procedure in which a needle is passing through a posterior wall of the left ventricle.

FIG. 3 depicts a subsequent step after the needle has passed through the posterior wall of the left ventricle, with a first tether attached.

FIG. 4 depicts a subsequent step, after the first tether has been exteriorized between the subject's ribs, and an expandable support has been attached to the first tether.

FIG. 5 depicts a subsequent step, after the expandable support has been approximated to the puncture location by retracting the first tether.

FIG. 6 is a detail of the first expandable support in position against an outside the posterior left ventricle wall after the first expandable support has been expanded.

FIG. 7 depicts a subsequent step, after a second needle and second tether have been advanced through the interventricular septum, then through the right ventricle wall, and then exteriorized between the subject's ribs.

FIG. 8 depicts a subsequent step in which an introducer sheath has been advanced over the second tether and into the right ventricle.

FIG. 9 depicts a subsequent step in which, after the tethers have been joined, a second expandable support 10 has been introduced into the right ventricle over the joined tethers.

FIG. 10 depicts a subsequent step in which the second expandable support has been expanded.

FIG. 11 depicts a subsequent step in which the second expandable support has been secured to the first expandable support via the joined tethers.

FIG. 12 depicts an alternative approach in which the second expandable support is positioned against an outer surface of the right ventricle.

FIG. 13 depicts an alternative approach in which the anterior tension is provided by affixing the first tether to one of the subject's ribs.

FIG. 14 depicts an alternative approach in which the second expandable support is positioned against an outer surface of the anterior wall of the subject's left ventricle.

FIG. 15 depicts yet another alternative approach in which the second expandable support is positioned within the right atrium in contact with the septal aspect of the tricuspid annulus above the ventricular septum.

FIG. 16 is a detailed view of the first expandable support in its expanded state.

FIG. 17 is a detailed view of the first expandable support in its initial collapsed state.

FIG. 18 depicts the heart of a healthy subject.

FIG. 19 depicts the heart of a subject who suffers from IMR.

FIG. 20 depicts the puncturing area on the tricuspid annulus, in the proximity of the mitral annulus, as viewed on the valves plain.

FIG. 21 depicts a catheter advanced through the vascular system into the RA, and a needle creating a diagonal direct puncture from the RA into the LV.

FIG. 22 depicts the catheter advanced into the LV and a needle creating a puncture through the ventricle wall between the papillary muscles.

FIG. 23 depicts a tether that has been pulled out of the patient's body between the ribs and attached to a first support plate.

FIG. 24 depicts the tether being pulled back, and the first support plate being inserted into the patient's body between the ribs and approximated to the outer wall of the LV.

FIG. 25 depicts a support bar after it has been advanced over the tether through the vascular system, into the RA adjacent to the puncture that was created between the RA and the LV.

FIG. 26 depicts the support bar location after it has been placed in position, as viewed on the valves plane.

FIG. 27 depicts the first support plate being pulled inward by the tether in direction D towards the support bar and secured to it under tension, to improve coaptation of the mitral valve.

FIG. 28 depicts the interconnection between a doubled tether and a support plate in an alternative embodiment.

FIG. 29 depicts the interconnection between the doubled tether and a support bar in the FIG. 28 embodiment.

FIG. 30 depicts the FIG. 28 embodiment when the support plate and the support bar are interconnected by the doubled tether.

FIG. 31 depicts adding another support plate to the FIG. 27 embodiment to support the RV wall under tension, to relieve the tethering forces of the tricuspid valve chordae tendinea.

FIG. 32 depicts the FIG. 31 embodiment as viewed on the valves plane.

FIG. 33 depicts adding a curved support member to the FIG. 27 embodiment to compress the tricuspid annulus under tension, to reduce or prevent dilatation of that annulus.

FIG. 34 depicts the FIG. 33 embodiment as viewed on the valves plane.

FIG. 35 depicts adding a curved support member to the FIG. 27 embodiment to compress the mitral annulus under tension, to reduce or prevent dilatation of the mitral annulus.

FIG. 36 depicts the FIG. 35 embodiment as viewed on the valves plane.

FIG. 37 depicts the components of the FIG. 27 embodiment, with labels for indicating corresponding dimensions.

FIG. 38 depicts the components of the FIG. 31 embodiment, with labels for indicating corresponding dimensions.

FIG. 39 depicts the components of the FIG. 33 embodiment, with labels for indicating corresponding dimensions.

FIG. 40 depicts an embodiment that is similar to the FIG. 27 embodiment, except that the straight support bar from the FIG. 27 embodiment is replaced with a curved support bar.

FIG. 41 depicts another embodiment in which a non-elongated support member has been advanced over a tether through the vascular system, into the RA adjacent to the puncture that was created between the RA and the LV.

FIG. 42 depicts the non-elongated support member of the FIG. 41 embodiment after it has been placed in position, as viewed on the valves plane.

FIG. 43 depicts an alternative embodiment in which the support plate is formed by solidifying a liquid precursor into a solid pad, at a stage before the support plate has been connected to the support bar using a tether.

Various embodiments are described in detail below with reference to the accompanying drawings, wherein like reference numerals represent like elements.

### DESCRIPTION OF THE PREFERRED EMBODIMENTS

### Section 1

The embodiments described in this section reduce the pulling force that the chordae exert on the leaflets by pulling the papillary muscles (to which the chordae are attached) in an anterior direction, which reduces the pulling force that the chordae exert on the leaflets. And notably, these embodiments use techniques that are much less invasive than the techniques that were used to install the prior art Coapsys device.

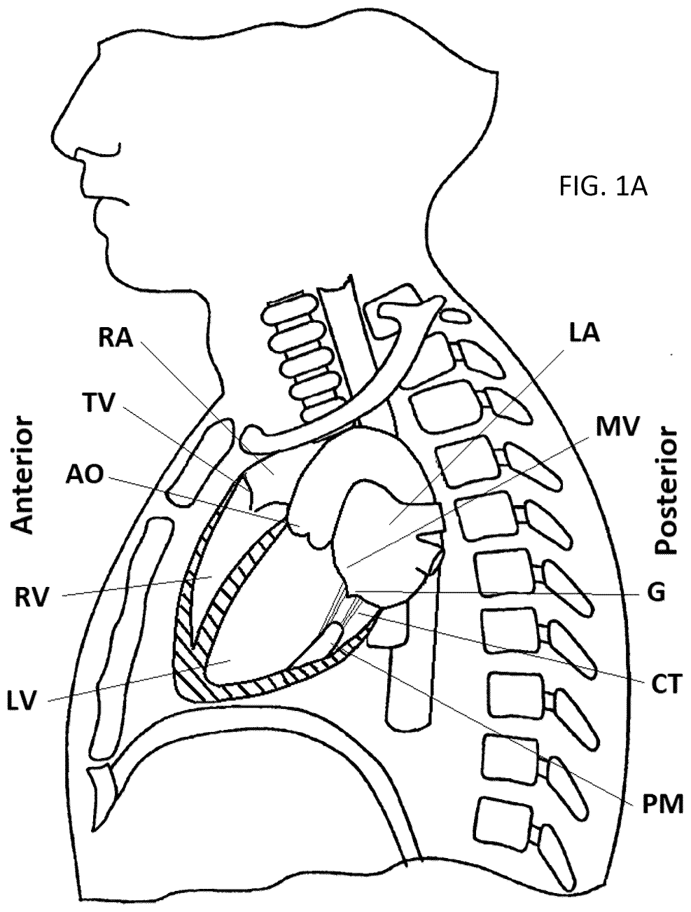

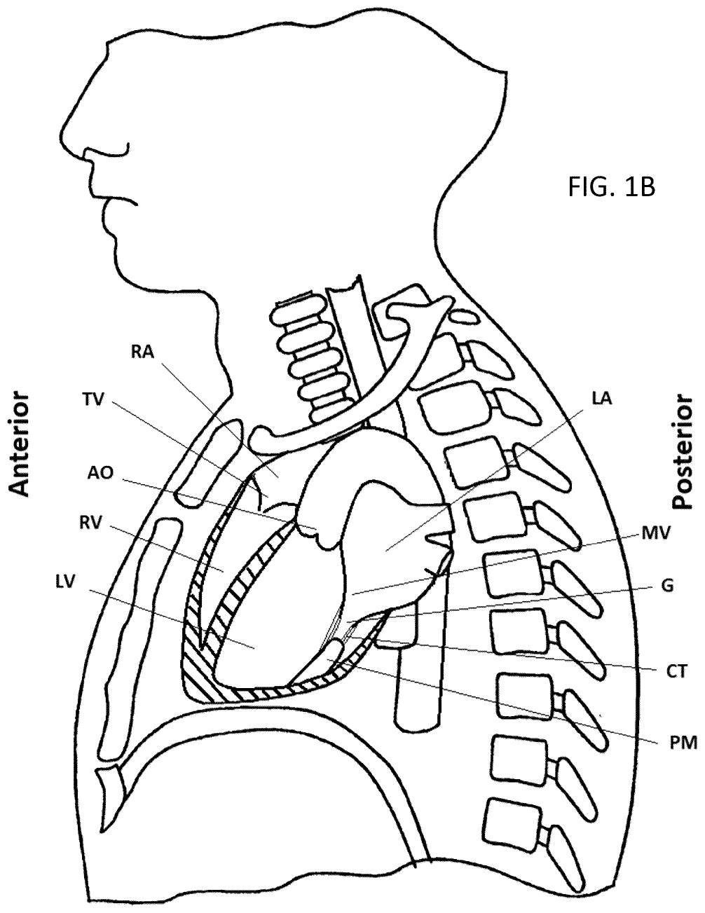

FIG. 1A is a longitudinal cross section of a healthy person's heart, on a plane passing through the heart between the papillary muscles of the mitral valve. The left ventricle LV, the right ventricle RV, the left atrium LA, the right atrium RA, the mitral valve MV, the aortic valve AO, and the tricuspid valve TV are all shown. Notably the chordae tendinea (CT) are attached to the posterior wall of the left ventricle via the papillary muscles (PM). And in the healthy subject depicted in FIG. 1A, the chordae do not prevent the leaflets of the mitral valve from closing.

FIG. 1B is a cross section of a subject who suffers from IMR caused by chronic changes of the left ventricle structure, along the same plane depicted in FIG. 1A. In this subject, the posterior wall of the left ventricle is positioned posteriorly with respect to the posterior wall in the healthy heart depicted in FIG. 1A. This shift increases the distance between the papillary muscles and the mitral valve. And because the leaflets of the mitral valve are tethered to the papillary muscles via the chordae, the chordae in this FIG. 1B subject will prevent the leaflets of the mitral valve from fully closing, which results in a gap G.

This application discloses a number of approaches for moving the posterior wall of the left ventricle in an anterior direction so that it more closely resembles the healthy person's heart depicted in FIG. 1A. All of the approaches described in this section rely on positioning a support structure at an outer surface of the posterior wall of the left ventricle, and using a tether that runs through the left ventricle to pull that support structure in an anterior direction.

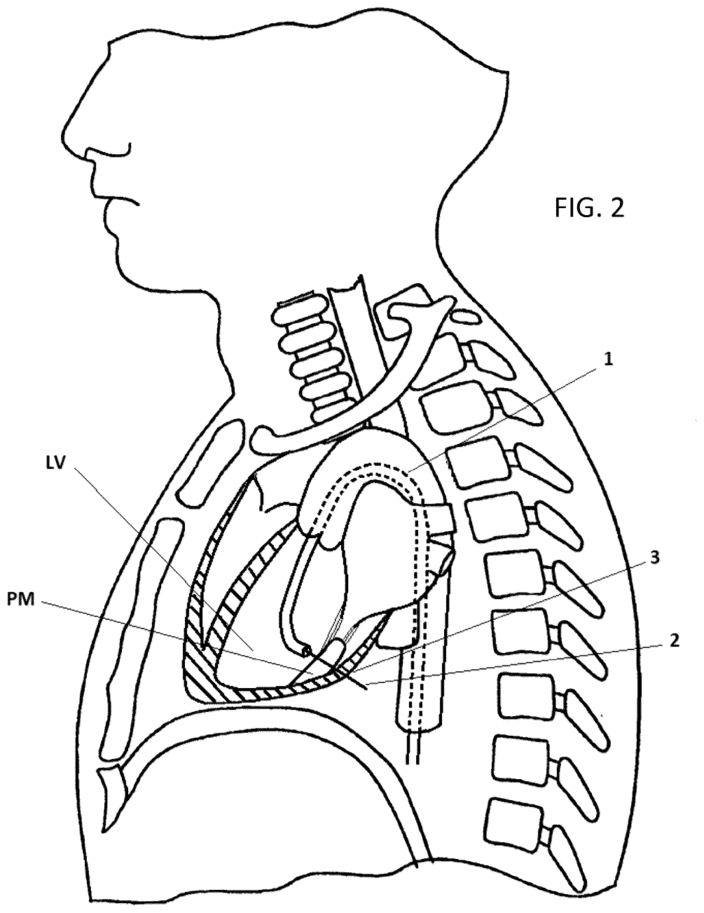

FIGS. 2-11 depict a first example of such an approach. It is envisioned (although not required) that the procedures described in this section will be implemented by two practitioners. The first practitioner could be, for example an interventional cardiologist who is experienced in accessing a subject's heart via a catheter that has been introduced through the subject's vasculature. And notably, the terminologies “distal” and “proximal” used in this section are with respect to the frame of reference of the first practitioner (i.e., the interventional cardiologist). The second practitioner could be, for example, a thoracic surgeon who knows how to access a subject's heart from outside of the heart using surgical techniques, including minimally invasive surgical techniques.

The first practitioner inserts a catheter 1 into the LV through the aorta as depicted in FIG. 2. the catheter 1 is manipulated (e.g., using echo and/or fluoro imaging) to a specific first location 3 in the posterior wall of the LV between the papillary muscles, and a first needle 2 (e.g., made of metal) is pushed through the posterior wall of the LV to puncture it and be exteriorized through it at a first location 3. The first needle 2 is advanced until a distal portion of the first tether 4 passes through the posterior wall of the LV and exits the heart.

Notably, because the first needle is introduced from within the heart, it will be relatively easy to line the first needle up between the papillary muscles (e.g., using echo and/or fluoro imaging). This is important because moving the papillary muscles will move the “roots” of the chordae in an anterior direction, in which case those “roots” will be closer to the leaflets of the mitral valve, which means that the chordae will either no longer prevent those leaflets from fully coapting, or at the very least improve coaptation of the leaflets.

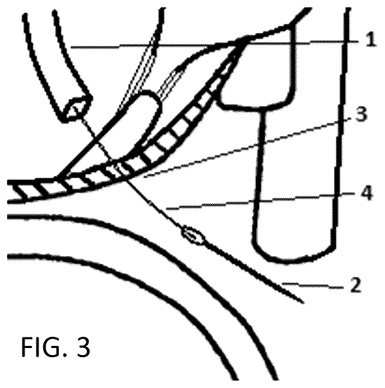

The first needle 2 is attached to a thin, strong polymeric first tether 4 as depicted in FIG. 3, and because of the very low profile of the puncture hole (along with the fact that immediately after the puncture is made, the puncture is plugged with the tether), no bleeding out of the ventricle will occur in the first puncture site 3. The first needle 2 in FIG. 3 could be, for example, short (e.g., 3-5 cm) and rigid, long (e.g., 100-200 cm) and flexible, or another type of needle may be used. The attachment detail of the first tether 4 to needle 2 may be implemented using any conventional approach, e.g., a commonly used needle and thread configuration as depicted in FIG. 3.

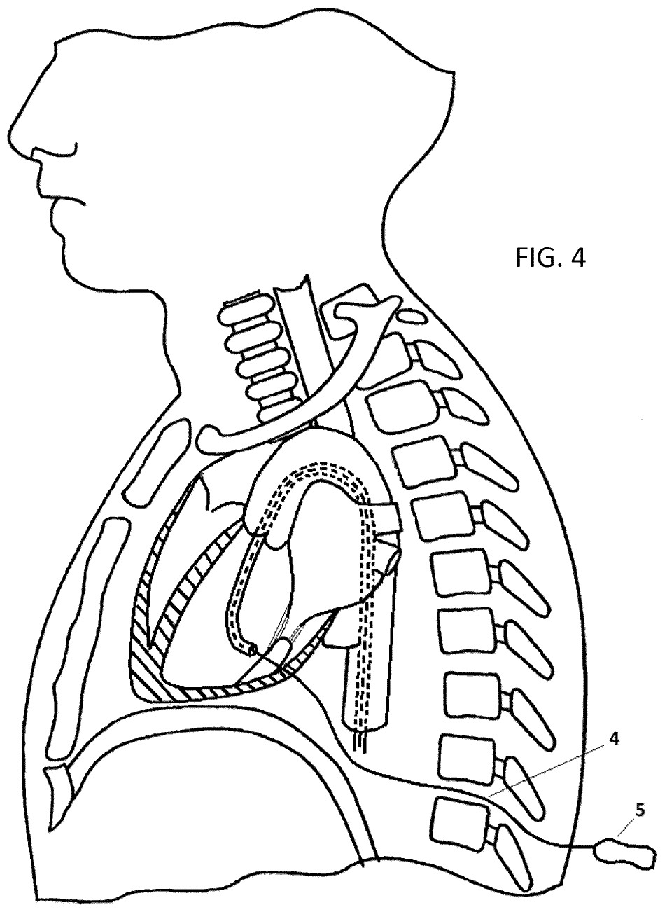

Using minimal invasive techniques as practiced in many thoracic lung surgeries, the second practitioner captures the first needle 2 (e.g., using forceps or a similar tool), as it emerges out of the ventricle wall. The first needle 2 and the first tether 4 are then advanced (e.g., pulled out) in a distal direction (with respect to the first practitioner) until a first portion of the first tether exits the subject's body e.g., through two adjacent ribs as depicted in FIG. 4.

Next, the second practitioner affixes a first expandable support 5 to the first tether 4 (e.g., using a knot, not shown, or any other suitable approach). At this stage, the first expandable support 5 is in its initial collapsed state.

The first practitioner then pulls the first tether 4 through catheter 1 in the proximal direction as depicted by the arrow in FIG. 5 to retract the first tether 4. Meanwhile, the second practitioner guides the first expandable support 5 so that it passes smoothly into the subject's body through the ribs (e.g., through a minimal invasive cut). This process continues until the first expandable support 5 reaches the outer surface of the posterior wall of the left ventricle adjacent to the first puncture location 3. The approximation of the first expandable support 5 to the first location 3 can be aided by the second practitioner's manual manipulation through the minimal invasive cut like in other surgical procedures.

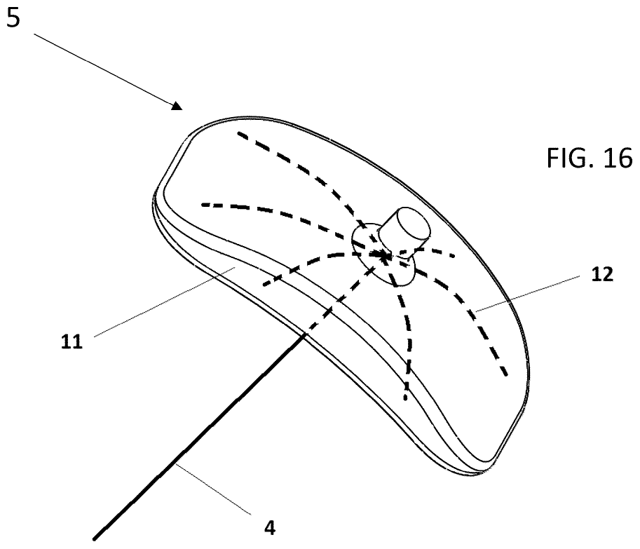

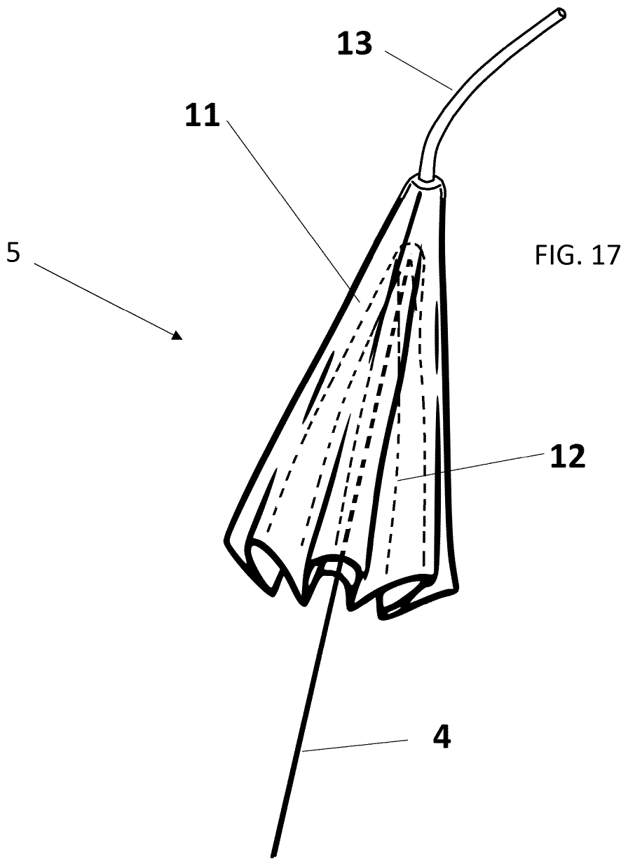

The first expandable support 5 can be made out of thin wall plastic bag 11 that can be collapsed as shown in FIG. 17 or expanded as shown in FIG. 16. Plastic bag 11 can be filled through a tube 13 with liquid polymer, that is configured to solidify when so desired, through a controlled chemical reaction like a two components thermosetting polymer (i.e., Epoxy or Acrylic). Optionally, an expandable scaffold 12 (e.g., made of flexible metallic arms) can be disposed within the plastic bag 11 much like umbrella arms. In its initial collapsed state, the plastic bag 11 can be constrained within a small diameter tube (not shown) to facilitate insertion into the body, and the optional arms 12 are also collapsed within the tube. When the first expandable support 5 reaches its destination, it exits the insertion tube, after which the arms 12 will expand to implement the spreading out of the plastic bag 11 into a flat shape as depicted in FIG. 16. The scaffold of arms 12 are attached to the first tether 4 (e.g., using a knot, clip, fastener, etc.).

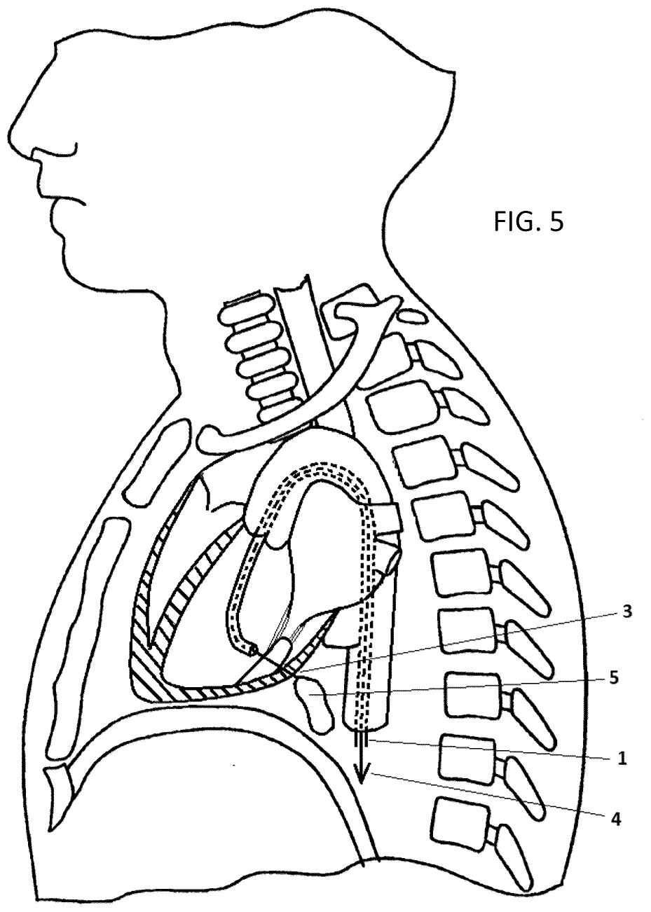

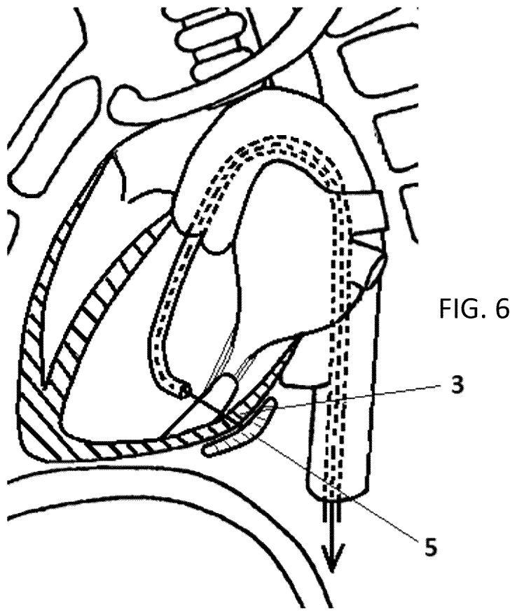

After the first expandable support 5 has expanded, as depict in FIG. 16 it is filled with medical grade solidifying polymer through tube 13, and upon solidification of the polymer the tube 13 can be removed. No liquid polymer leak will occur out of plastic bag 11 when the filling tube is removed as the liquid polymer will have been transformed into a solid. At this point in the procedure, we have a single expandable support 5, in its expanded state, positioned just outside the subject's heart, at a location that is within 3 cm (or more preferably within 2 cm) of the midpoint between the center of the two papillary muscles, with a region of contact between the first expandable support 5 and the outer surface of the posterior wall of the LV, as seen in FIG. 6. In some embodiments, this region of contact has an area of at least 2 cm². In some embodiments, this region of contact has an area of at least 5 cm 2. In some embodiments, this region of contact has an area of 6-15 cm².

In the next step of the procedure, as depicted in FIG. 7, a second needle (not shown) is introduced into the left ventricle (e.g., as described above for the first needle 2). The second needle is affixed to a second tether 6. The first practitioner pushes the second needle through the septum of the heart at a second location 7 and subsequently pushes it through an outer wall of the subject's right ventricle at location 8 until a distal portion of the second tether 6 passes through the outer wall of the right ventricle and exits the heart. The second tether 6 is then advanced in a distal direction until a first portion of the second tether 6 exits the subject's body (e.g., between two ribs). This step of the procedure at the anterior aspect of the heart can be performed as described above for the first tether at the posterior aspect of the heart.

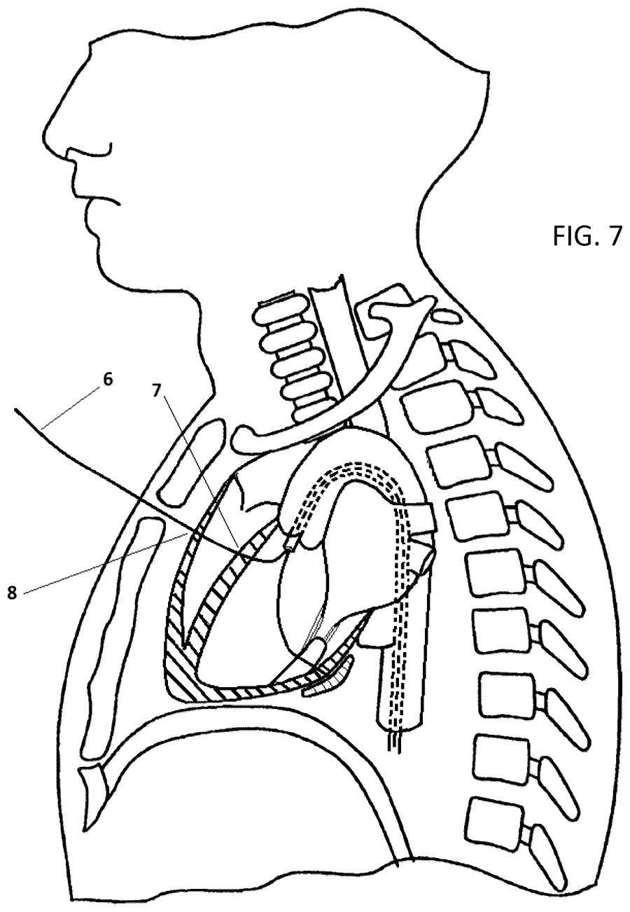

An introducer sheath 9 is then advanced over the second tether 6 between the subject's ribs into the RV through the puncture at location 8 as depicted in FIG. 8. In some embodiments, the second practitioner advances a second expandable support 10 over the second tether 6 and into the subject's body through introducer sheath 9 (i.e., in a proximal direction with respect to the first practitioner) into the right ventricle.

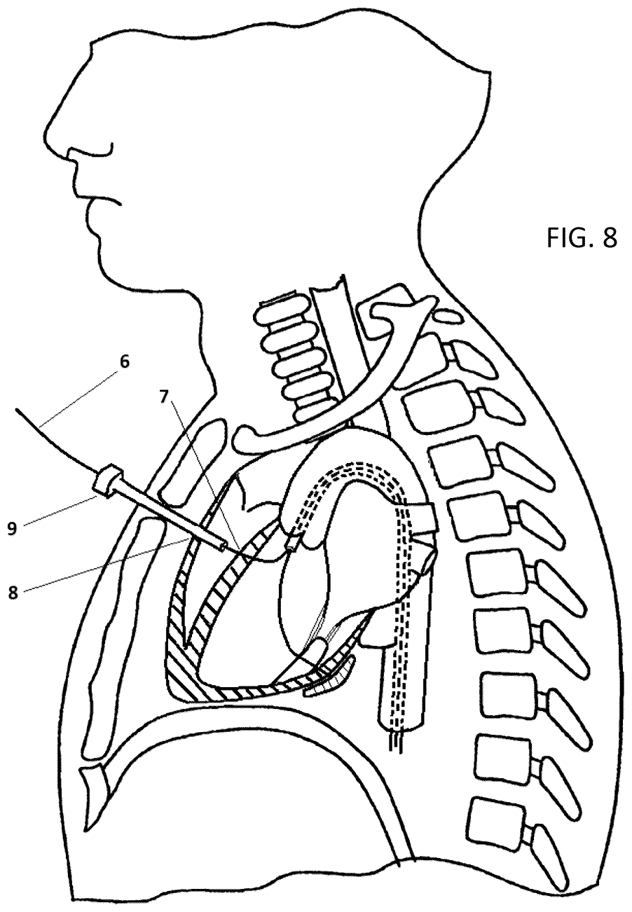

In some embodiments, after the second expandable support 10 enters the right ventricle and reaches the septum adjacent to the second location 7, the second tether 6 is joined to the first tether 4 (e.g., using a knot, a clip, etc.) at this point in the procedure, resulting in the joined tether configuration depicted in FIG. 9. The second expandable support structure 10 is then expanded (e.g., as described above in connection with the first expandable support structure 5) so that a region of contact between the second expandable support structure 10 and the septum is formed at location 7, as depicted in FIG. 10.

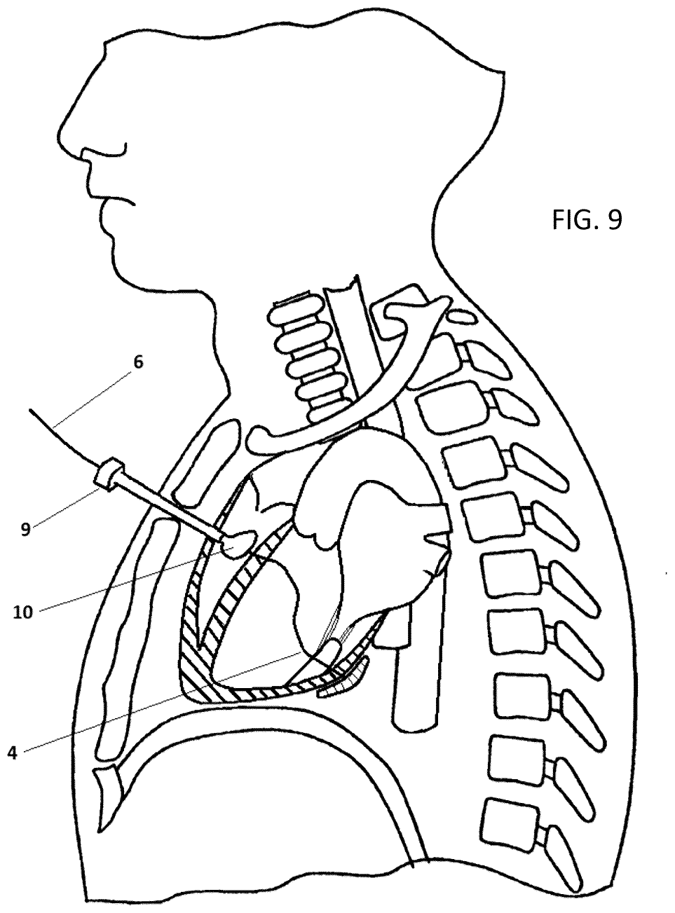

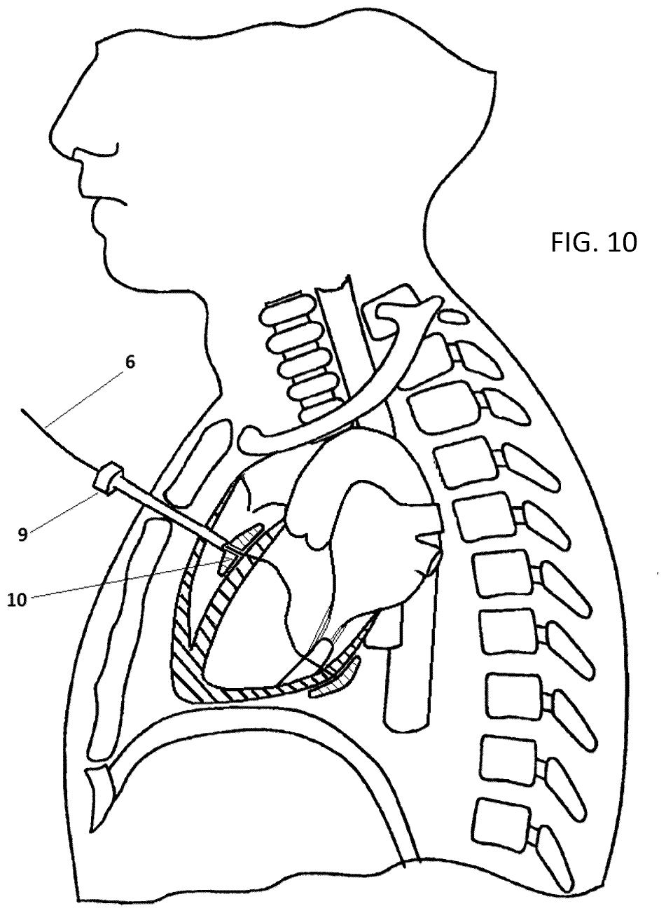

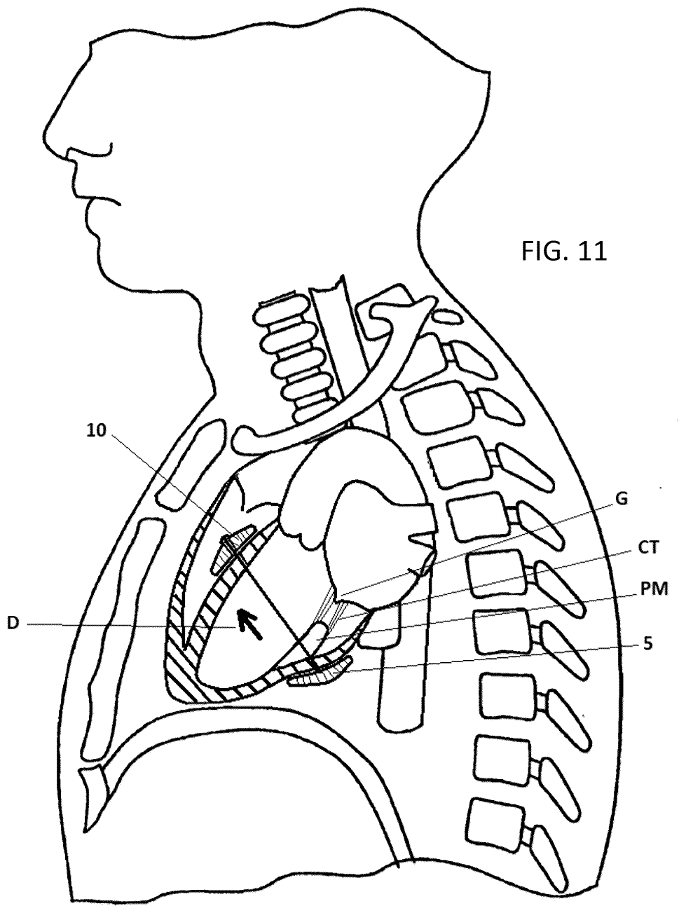

In these embodiments, the second tether 6 can pass freely through the second expandable support structure 10 at this stage in the procedure, so a pull force can be applied through it to the first expandable support 5 on the posterior aspect of the heart while bottoming the second expandable support structure with introducer sheath 9. The pull force applied to the joined tether 6+4 acts to approximate the first expandable support 5 in the direction of the second expandable support 10, and to create a directional deformation vector D to the posterior wall of the LV. The second expandable support 10 is then affixed to the second tether 6 (e.g., using a knot, clamp, clip, fastener, etc.), thereby securing the first tether 4 to a portion of the subject's body that is located anteriorly with respect to the left ventricle via an intervening component (i.e., the second tether 6), as depicted in FIG. 11.

Note that the first tether 4 may be secured to the portion of the subject's body that is located anteriorly (e.g., the interventricular septum) either directly or indirectly (i.e., through one or more intervening components, including but not limited to the second tether 6). For example, the second tether can be attached to the first tether in a manner that establishes tension in the first tether, wherein the tension pulls the first expandable support structure towards the second expandable support structure. Alternatively, if the joined tether 6+4 is formed using a relatively long section of the first tether 4 and a relatively short section of the second tether 6, the entire second tether 6 may be pulled out distally beyond the second expandable support 10, in which case the second expandable support 10 would be affixed directly to the first tether 4 (e.g., using a knot, clamp, clip, fastener, etc.), thereby directly securing the first tether 4 to a portion of the subject's body that is located anteriorly with respect to the left ventricle.

The posterior LV wall deformation creates a traction displacement to the PM in the same direction, which reverses and loosens the tethering forces on the chordae and allows the mitral valve leaflets better coaptation, to repair the mitral valve insufficiency.

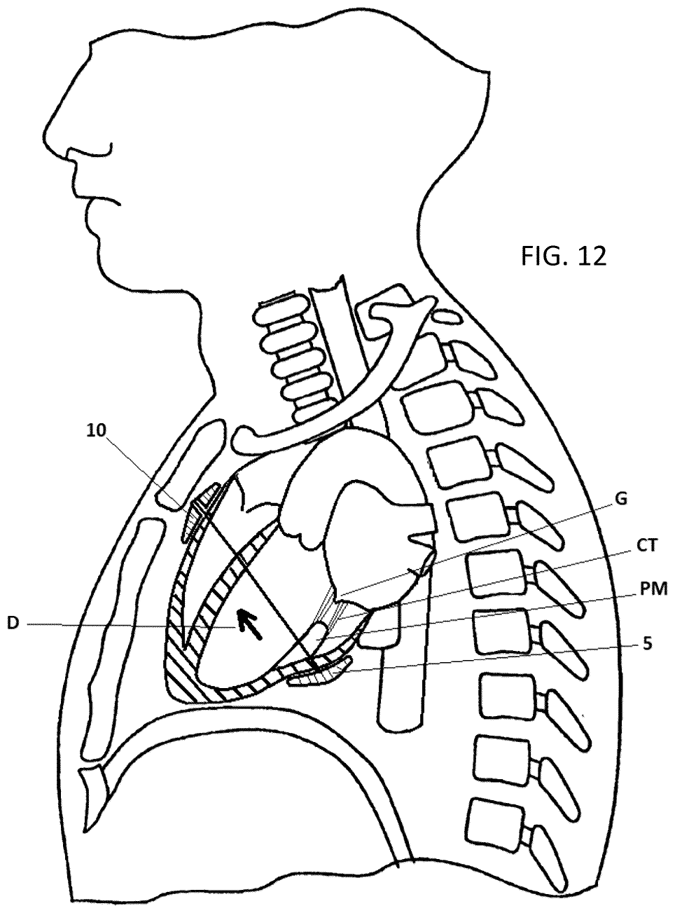

In alternative embodiments, the second expandable support 10 may be positioned at a different anatomic location. A number of examples are described below in connection with FIGS. 12-15.

FIG. 12 depicts one alternative location for the second expandable support 10 outside the RV on the anterior aspect of the heart. The second expandable support 10 is installed using a procedure similar to the procedure described above in connection with FIGS. 7-11, but with the second expandable support 10 positioned at an outer surface of the right ventricle.

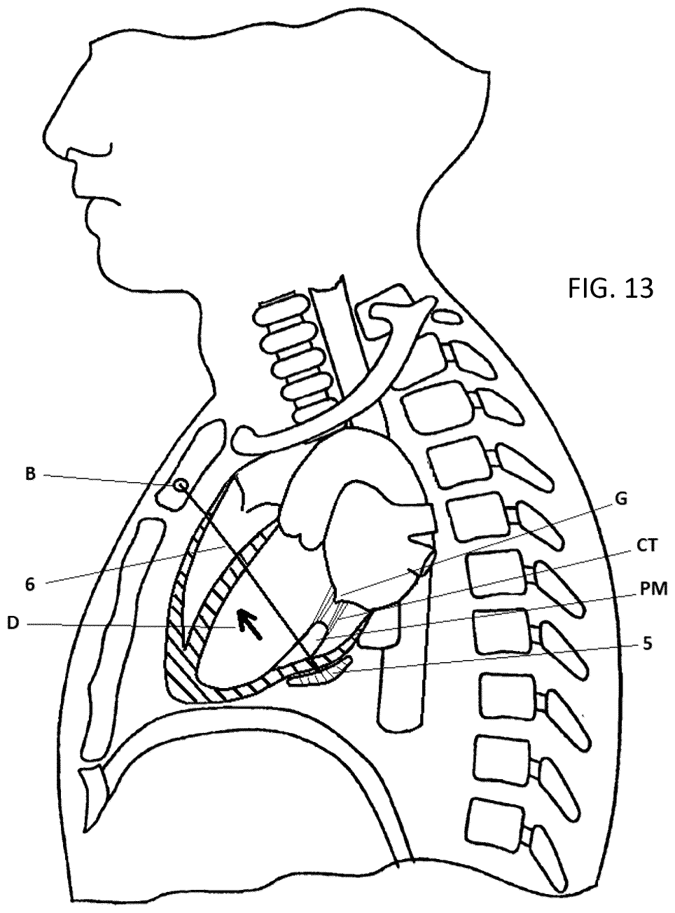

FIG. 13 depicts another option for anchoring the anterior hinge point, by securing the second tether 6 to a rib or a bone in the subject's chest. The tether can be secured in this location B by tying it around a rib, or by attaching it with a screw to a bone, or by any other means. Notably, this embodiment does not rely on a second expandable support.

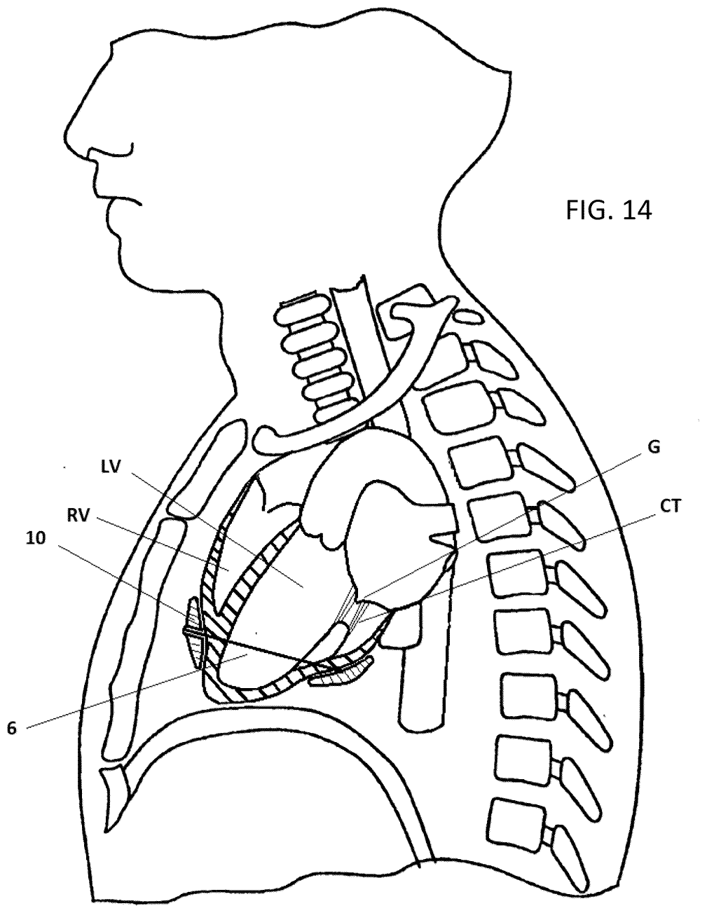

FIG. 14 depicts another alternative location for the second expandable support 10 outside the LV (and RV) on the anterior aspect of the heart. The second expandable support 10 is installed using a procedure similar to the procedure described above in connection with FIGS. 7-11, but with the second expandable support 10 positioned at an outer surface of the anterior wall of the left ventricle.

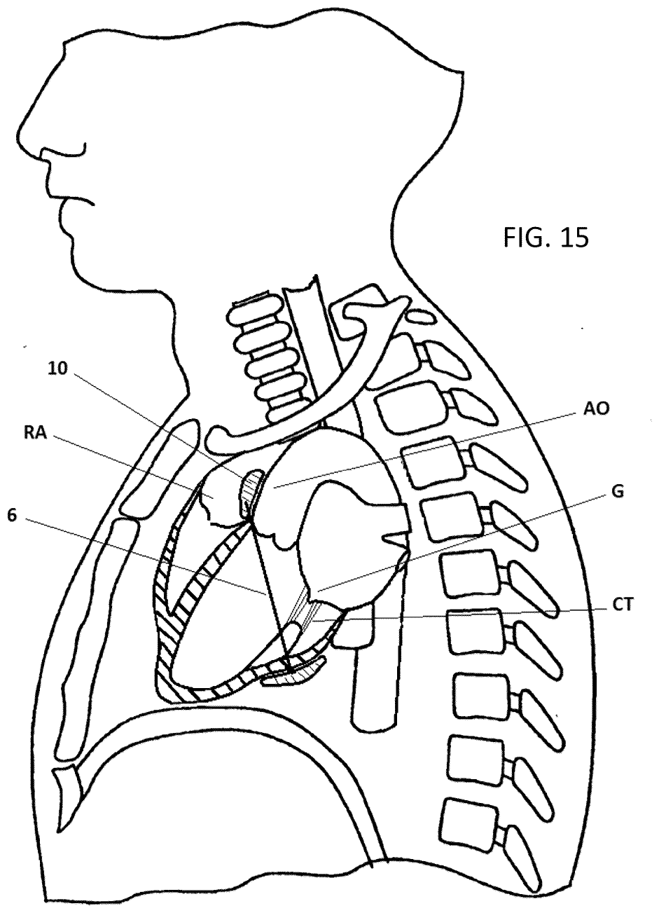

Finally, FIG. 15 depicts yet another alternative location for the second expandable support 10 inside the right atrium, in contact with the septal aspect of the tricuspid annulus above the ventricular septum. The second expandable support 10 is installed using a procedure similar to the procedure described above in connection with FIGS. 7-11, but with the second expandable support 10 positioned within the right atrium in contact with the tricuspid annulus and the external surface of the aortic arch at the location of the membranous septum and the right fibrous trigone. This position can be particularly advantageous because the geometry is such that the “roots” of the chordae will be urged in a direction that can more effectively ameliorate the tension that the chordae exert on the leaflets of the mitral valve.

Installing the second expandable support 10 at this position may be implemented by having the first practitioner push the second needle through the anterior wall of the LV so that it enters the RA just above the tricuspid annulus, and subsequently pushing the needle through an outer wall of the subject's right atrium until a distal portion of the second tether passes through the outer wall of the right atrium and exits the heart. The second tether is then advanced in a distal direction until a first portion of the second tether exits the subject's body (e.g., between two ribs).

An introducer sheath is then advanced over the second tether between the subject's ribs into the RA through the puncture in the outer wall of the RA. In some embodiments, the second practitioner advances the second expandable support 10 over the second tether and into the subject's body through an introducer sheath (i.e., in a proximal direction with respect to the first practitioner) into the right atrium. After the second expandable support 10 enters the right atrium and reaches the posterior wall of the RA, just above the tricuspid annulus, the second tether is joined to the first tether (e.g., using a knot, a clip, etc.) at this point in the procedure, resulting in a joined tether configuration. The second expandable support structure 10 is then expanded (e.g., as described above in connection with the first expandable support structure 5) so that a region of contact between the second expandable support structure 10 and the surface of the tricuspid annulus formed, adjacent to the posterior wall of the RA. The rest of the procedure similar to the procedure described above in connection with FIGS. 10-11.

### Section 2

The Embodiments Described in this Section Reduce the Pulling Force that the chordae exert on the leaflets by pulling the papillary muscles (to which the chordae are attached) toward the right atrium, which reduces the pulling force that the chordae exert on the leaflets. And these embodiments also use techniques that are much less invasive than the techniques that were used to install the prior art Coapsys device.

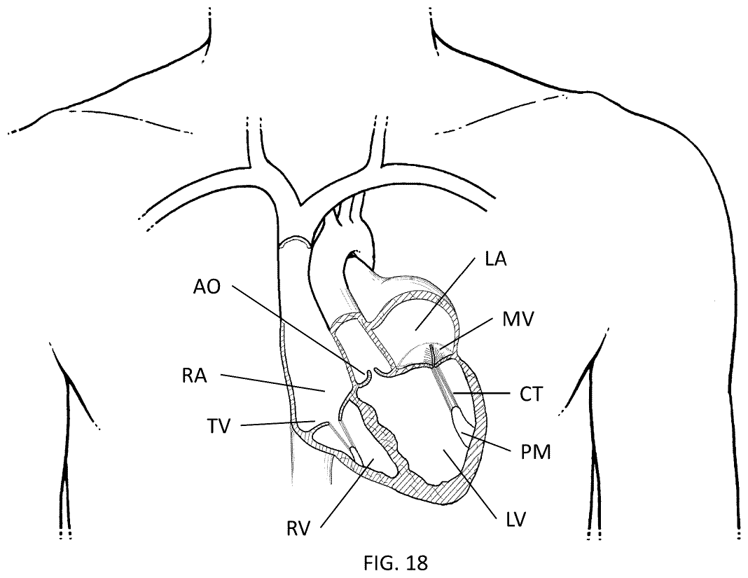

FIG. 18 is an anterior cutaway view of a healthy person's heart in which the left ventricle LV, the right ventricle RV, the left atrium LA, the right atrium RA, the mitral valve MV, the aortic valve AO, and the tricuspid valve TV are visible. Two chordae tendinea (CT) are attached to the posterior wall of the left ventricle via the papillary muscles (PM). And in the healthy subject depicted in FIG. 18, the chordae do not prevent the leaflets of the mitral valve from closing.

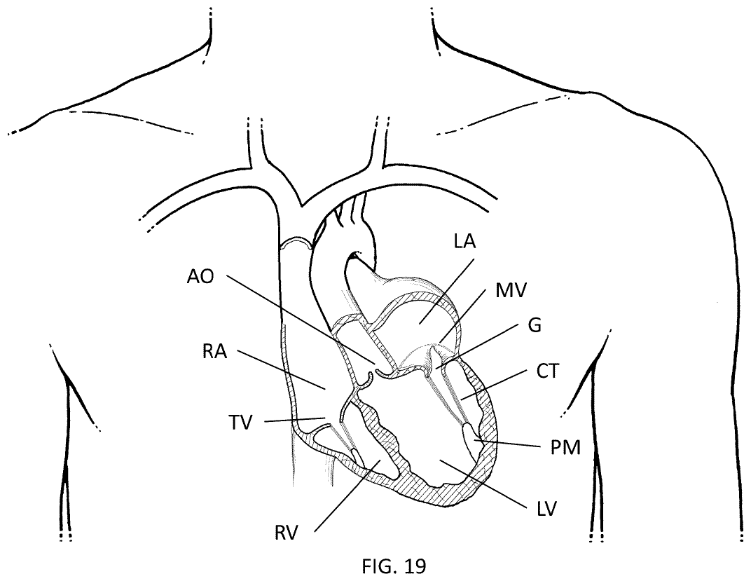

FIG. 19 is a cross section of a subject who suffers from IMR caused by chronic changes of the left ventricle structure, along the same cutaway view depicted in FIG. 18. In this subject, the posterior wall of the left ventricle is dilated, causing the papillary muscles to be shifted more posteriorly with respect to the posterior wall in the healthy heart depicted in FIG. 18. This shift increases the distance between the papillary muscles and the mitral valve leaflets. And because the leaflets of the mitral valve are tethered to the papillary muscles via the chordae, the chordae in this FIG. 19 subject will prevent the leaflets of the mitral valve from fully closing, which results in a gap G.

This application discloses a number of approaches for moving the posterior wall of the left ventricle so that it more closely resembles the healthy person's heart depicted in FIG. 18. All of the approaches described in this section rely on positioning a support plate against an outer wall of the left ventricle, and using a tether that runs through the left ventricle to pull that support plate diagonally towards the right atrium.

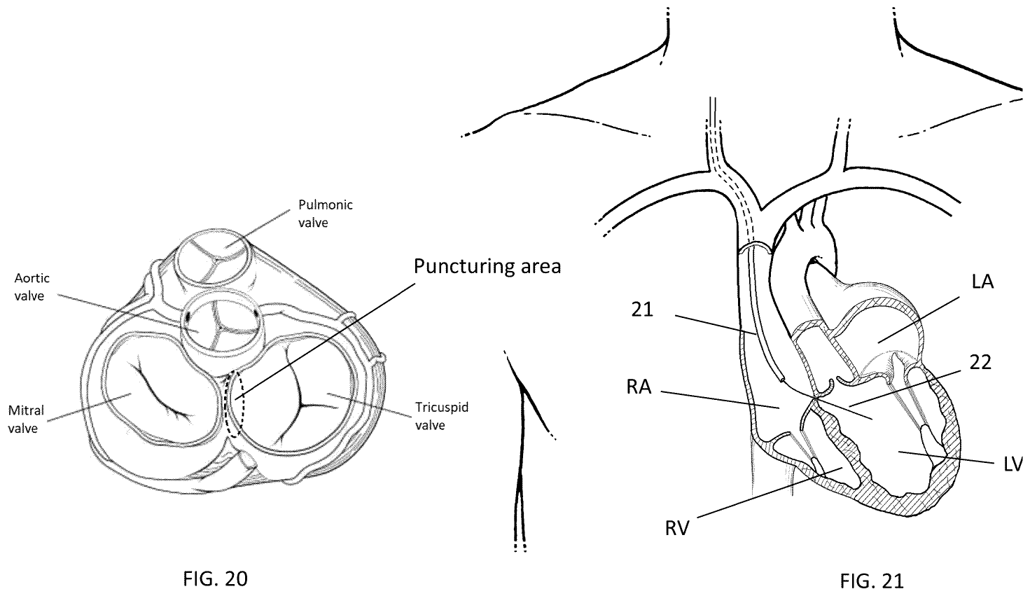

FIGS. 20-27 depict a first example of such an approach. It is envisioned (although not required) that the procedures described in this section will be implemented by two practitioners. The first practitioner could be, for example an interventional cardiologist who is experienced in accessing a subject's heart via a catheter that has been introduced through the subject's vasculature. And notably, the terminologies “distal” and “proximal” used in this section are with respect to the frame of reference of the first practitioner (i.e., the interventional cardiologist). The second practitioner could be, for example, a thoracic surgeon who knows how to access a subject's heart from outside of the heart using surgical techniques, including minimally invasive surgical techniques.

The first practitioner inserts a catheter 21 through the vascular system into the right atrium (with access obtained, e.g., via the jugular vein or another approach). The catheter 21 is manipulated (e.g., using echo and/or fluoro imaging) to the vicinity of the puncturing area depicted in FIG. 20, and a first needle 22 (e.g., made of metal) is advanced to protrude from the catheter 21. The first needle 22 is manipulated and advanced to create a direct diagonal puncture from the right atrium into the left ventricle as shown in FIG. 21. The puncture is performed at a portion of the tricuspid annulus that is directly above the interventricular septum, between the mitral and the tricuspid annulus as shown in FIG. 20. And this puncture serves as a first passageway between the right atrium and the left ventricle.

The catheter 21 is then further advanced through the first passageway into the left ventricle, and the first needle 22 is advanced through the catheter 21 to a location in the outer wall of the LV that is near at least one of the left-ventricular papillary muscles, and more preferably between the two left-ventricular papillary muscles. The first needle 22 is then pushed through the outer wall of the LV to puncture it and be exteriorized through that wall, as depicted in FIG. 22. The puncture in the outer wall of the LV serves as a second passageway.

Notably, because the first needle 22 is introduced from within the heart, it will be relatively easy to line the first needle up between the papillary muscles (e.g., using echo and/or fluoro imaging). This is important because moving the papillary muscles will move the “roots” of the chordae to be closer to the leaflets of the mitral valve, which means that the chordae will either no longer prevent those leaflets from fully coapting, or at the very least improve coaptation of the leaflets.

The first needle 22 is attached to a thin, strong polymeric tether 23, and because of the very low profile of the puncture hole in the LV wall (along with the fact that immediately after the puncture is made, the puncture is plugged with the tether), no bleeding out of the ventricle will occur in the second puncture site. The first needle 22 in FIGS. 20-22 could be, for example, short (e.g., 3-5 cm) and rigid, longer (e.g., 10-20 cm) and flexible, or another type of needle may be used. The attachment detail of the tether 23 to needle 22 may be implemented using any conventional approach.

An access is created between the patient's ribs into the mediastinum cavity using regular surgical techniques as often used in lung surgery procedures or using minimal invasive techniques as practiced in many thoracic lung surgeries. The second practitioner captures the first needle 22 (e.g., using forceps or a similar tool), e.g., as it emerges out of the ventricle wall. The first needle 22 and the tether 23 are then advanced (e.g., pulled out) in a distal direction (with respect to the first practitioner) until a first portion of the tether 23 exits the subject's body e.g., through two adjacent ribs.

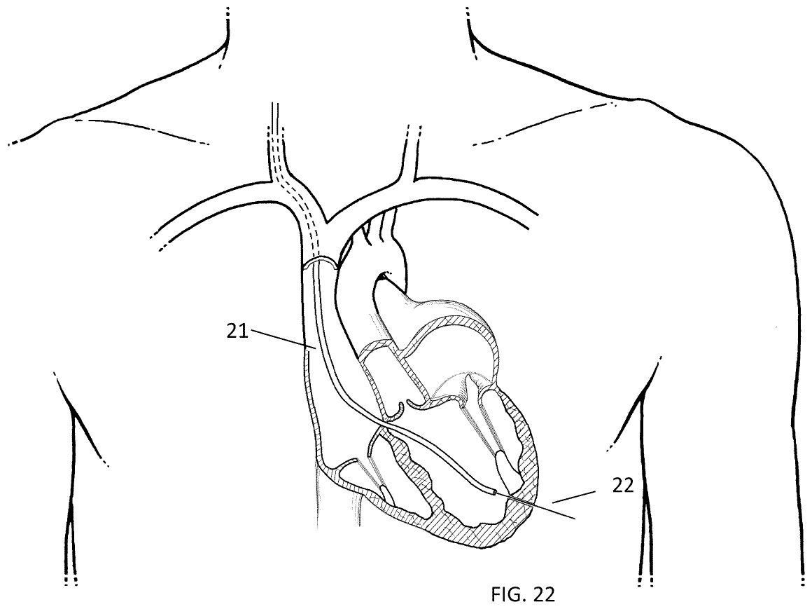

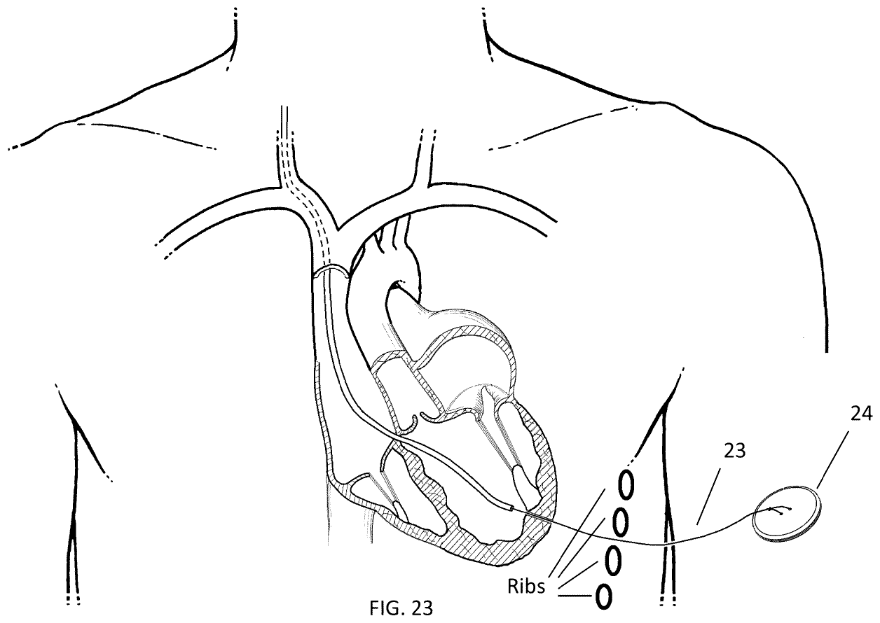

The second practitioner then secures a first support plate 24 to the tether 23 as depicted in FIG. 23 (e.g., using a knot or any other suitable approach).

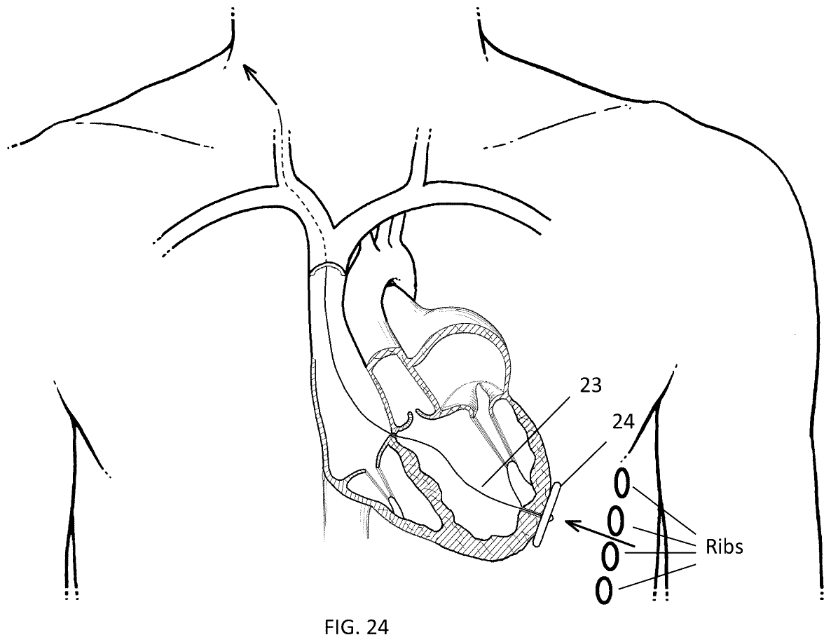

The first practitioner then pulls the tether 23 through catheter 21 in the proximal direction to retract the tether 23. Meanwhile, the second practitioner guides the first support plate 24 so that it passes smoothly into the subject's body through the ribs (e.g., by rotating the first support plate 24 sideways so that it will fit between the ribs through a minimal invasive cut into the mediastinum cavity). This process continues until the first support plate 24 reaches the outer surface of the outer wall of the left ventricle adjacent to the second passageway (which is in the vicinity of the left-ventricular papillary muscles), as depicted in FIG. 24. The approximation of the first support plate 24 to the second passageway can be aided by the second practitioner's manual manipulation.

In some embodiments, the surface of the first support plate 24 that faces the outer wall of the left ventricle is completely flat. But in alternative embodiments, that surface can be either convex or concave curved to some extent.

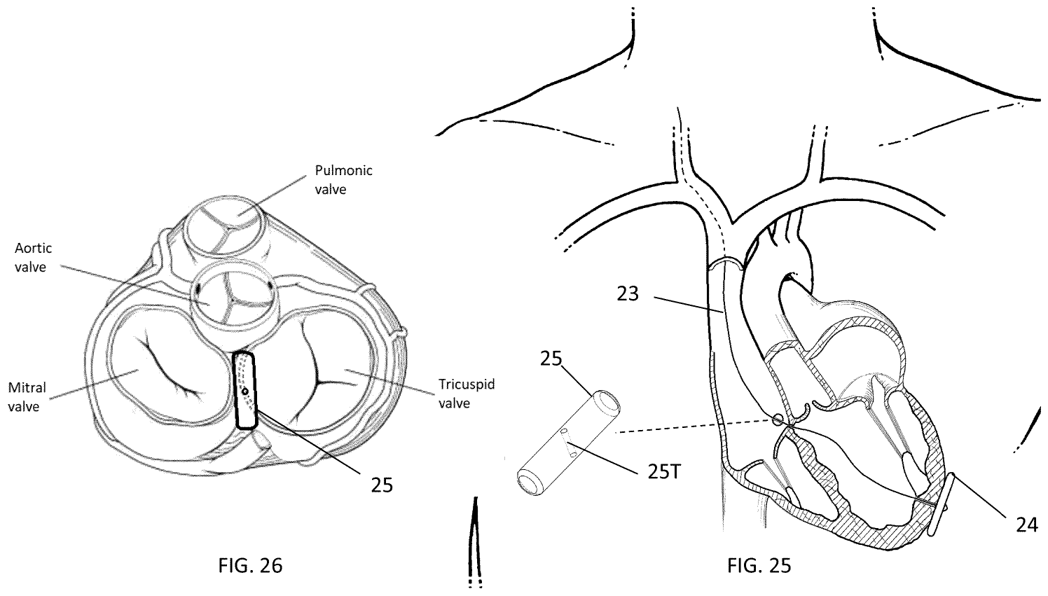

A first support member (which, in the embodiments described below in connection with FIGS. 25-39, is the support bar 25) is then advanced over the tether 23 via the vascular system until it contacts the tissue at the site of the puncture between the right atrium and the left ventricle (as depicted in FIG. 25 and FIG. 26). In some preferred embodiments, the support bar 25 is cylindrical. But in alternative embodiments, the support bar 25 can have a different shape (e.g., a half-cylinder, or the shape described below in connection with FIG. 40). The shape of the support bar 25 should be such that the support bar can be delivered via a catheter.

The first support plate 24 and the support bar 25 can be made from a suitable molded or machined biocompatible plastic (including but not limited to PEEK, PC, PSU, and/or ABS). And the tether 23 can be a suitable braided plastic with a high tensile strength (including but not limited to nylon and/or ultra-high-molecular-weight polyethylene).

The location of support bar 25 after it has been placed in position is depicted in FIG. 25 (a cross section of the heart chambers' view) and in FIG. 26 (cross section of heart valves plane). More specifically, the support bar 25 is positioned within the right atrium in contact with a septal portion of the tricuspid annulus, and adjacent to the first passageway.

In some preferred embodiments, the support bar 25 has a through-hole 25T, and the tether 23 is threaded through this through hole 25T before the support bar 25 is delivered into the right atrium by advancing it over the tether 23. In the illustrated embodiment, the through hole 25T is in the center of the support bar 25. But in alternative embodiments, the through hole 25T could be offset from the center e.g., by up to 25% of the support bar's length.

A pull force in the proximal direction is then applied on the tether 23 while maintaining the position of support bar 25 in contact with the tissue. The pull force may be applied by pulling a portion of the tether 23 that remains outside of the subject's body in a proximal direction. And the position of the support bar 25 may be maintained by using a catheter-based tool to push the support bar 25 against the tricuspid annulus. This creates tension in the tether 23, which acts to pull the first support plate 24 towards the support bar 25, as depicted by the arrow D in FIG. 27.

The anatomical location at the contact area of support bar 25 is a central part of the fibrotic skeleton of the heart, which is much more rigid relative to the heart muscle tissue at the anatomical location of the first support plate 24. Therefore, the tension on tether 23 acts to apply traction in direction D to the segment of the relatively soft ventricle wall which is the base of the papillary muscles as shown in FIG. 27. The directional traction of the papillary muscles acts to relieve tension from the mitral valve chordae tendinea and allow better coaptation of the leaflets and thereby improve the valve functioning.

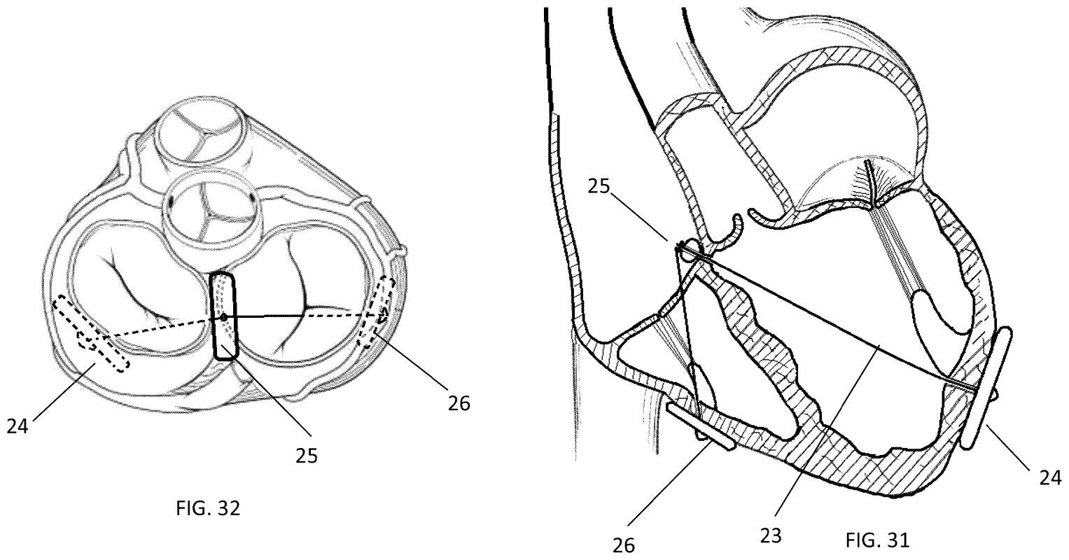

When the desired improvement in the valve functioning has been achieved, tether 23 is secured to the support bar 25 e.g., using a knot or a clip (not shown) to maintain tension between the first support plate 24 and the support bar 25 and preserve the improved hemodynamics. In some embodiments, the procedure is nearly complete after the knot has been tied or the clip has been installed. In these embodiments, the portion of the tether 23 that is proximal with respect to the knot or clip is cut off and discarded, at which point the procedure is complete. In other embodiments, additional components can be installed after the knot/clip has been tied/installed. Examples of these embodiments are described below in connection with FIGS. 31-36. In these embodiments, the portion of the tether 23 that is proximal with respect to the knot/clip is not cut off. Instead, that portion is used to connect to the additional components, as described below in connection with FIGS. 31-36.

The FIG. 27 embodiment can be particularly advantageous because the geometry is such that the “roots” of the chordae will be urged in a direction that can effectively ameliorate the tension that the chordae exert on the leaflets of the mitral valve.

Note that the embodiments described above in connection with FIGS. 20-27 rely on a single tether 23 that is (a) initially advanced from the right atrium into the left ventricle, (b) subsequently advanced from the left ventricle until it exits the subject's heart, (c) subsequently advanced until it exits the subject's body, and (d) subsequently retracted until the first support plate reaches an outer surface of the outer wall of the left ventricle adjacent to the second passageway.

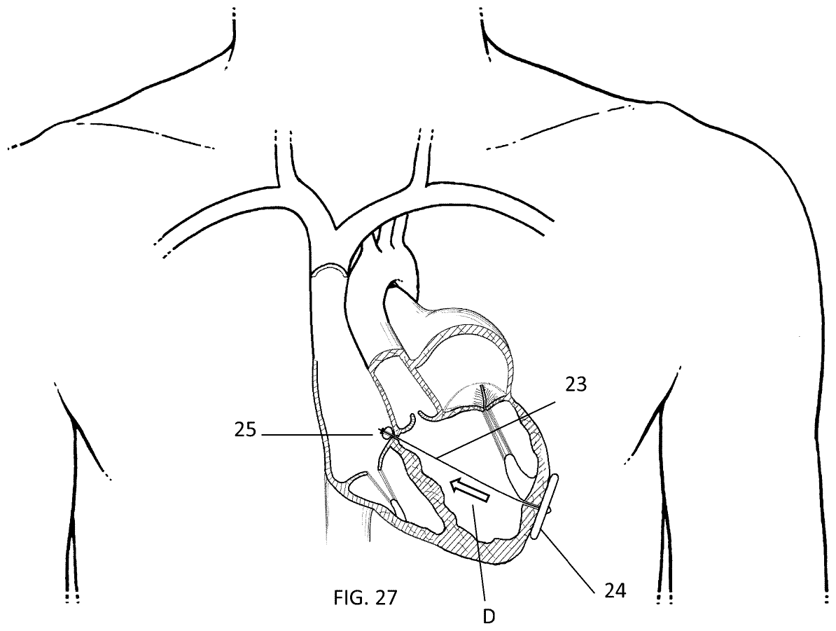

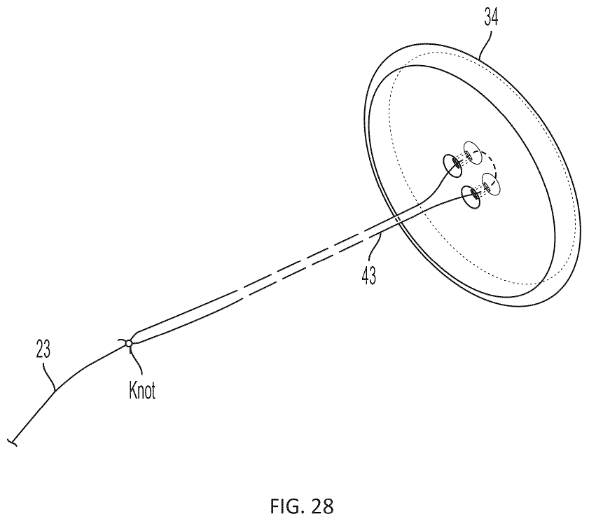

But in an alternative set of embodiments, distinct tethers can be used for the advancing function and the retracting function. These embodiments begin in the same manner described above in connection with FIGS. 20-23, up to and including the point where a first portion of the tether 23 exits the subject's body e.g., through two adjacent ribs. But after this initial portion of the procedure, instead of securing the first support plate 24 to the original tether 23 as depicted in FIG. 23, a second doubled tether 43 is looped through and back two through-holes in a support plate 34, as depicted in FIG. 28.

The proximal end of the doubled tether 43 is affixed to the distal end of the original tether 23, and the original tether 23 is used to pull the doubled tether 43 in a proximal direction all the way out of the subject's body via the original catheter 21 that was used to introduce the needle 22 (as described above in connection with FIG. 21). More specifically, the proximal end of the doubled tether will first be pulled in a proximal direction into the subject's left ventricle (via the second passageway), subsequently be pulled in a proximal direction into the subject's right atrium (via the first passageway), and subsequently be pulled in a proximal direction out of the subject's body via the jugular access to the right atrium.

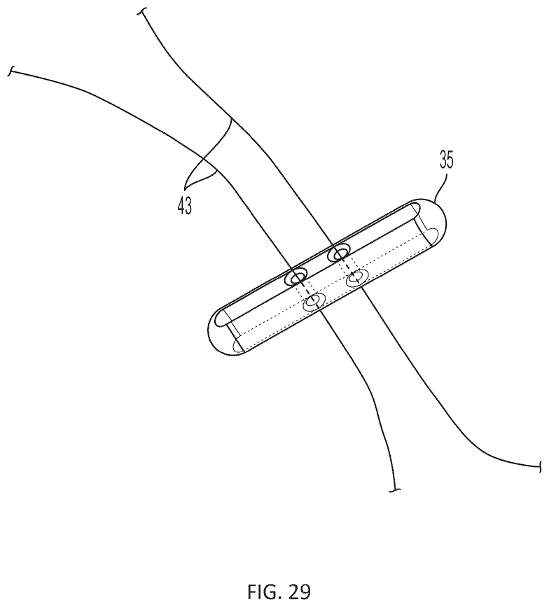

After the proximal end of the doubled tether 43 has exited the subject's body, the first practitioner threads each of the two ends of the doubled tether 43 through a respective hole in a support bar 35, as depicted in FIG. 29. (Note that in this embodiment, the support bar 35 serves as the first support member.) The first practitioner then advances the support bar 35 in a distal direction over the doubled tether 43 via the subject's vasculature until the support bar 35 enters the right atrium and contacts the tissue at the site of the puncture between the right atrium and the left ventricle. Meanwhile, the distal end of the doubled tether 43 (which is looped through the two through-holes in the support plate 34) as well as the support plate 34 itself both remain outside the subject's body.

In some preferred embodiments, the support bar 35 is cylindrical. But in alternative embodiments, the support bar 35 can have a different shape (e.g., a half-cylinder, or the shape described below in connection with FIG. 40). The shape of the support bar 35 should be such that the support bar can be delivered via a catheter. The location of the support bar 35 after it has been placed in position is similar to the position of the support bar 25 described above in connection with FIGS. 25 and 26. More specifically, the support bar 35 is positioned within the right atrium in contact with a septal portion of the tricuspid annulus, and adjacent to the first passageway.

The first practitioner then pulls the doubled tether 43 through catheter 21 in the proximal direction to retract the doubled tether 43 and pull the support plate 34 in a proximal direction. Meanwhile, the second practitioner guides the support plate 34 so that it passes smoothly into the subject's body through the ribs (e.g., by rotating the support plate 34 sideways so that it will fit between the ribs through a minimal invasive cut into the mediastinum cavity). This process continues until the support plate 34 reaches the outer surface of the outer wall of the left ventricle adjacent to the second passageway (which is in the vicinity of the left-ventricular papillary muscles), which is similar to the position of the support plate 24 depicted in FIG. 24. The approximation of the support plate 34 to the second passageway can be aided by the second practitioner's manual manipulation.

In some embodiments, the surface of the support plate 34 that faces the outer wall of the left ventricle is completely flat. But in alternative embodiments, that surface can be either convex or concave curved to some extent.

A pull force in the proximal direction is then applied on the doubled tether 43 while maintaining the position of support bar 35 in contact with the tissue. The pull force may be applied by pulling a portion of the doubled tether 43 that remains outside of the subject's body in a proximal direction. And the position of the support bar 35 may be maintained by using a catheter-based tool to push the support bar 35 against the tricuspid annulus. This creates tension in the doubled tether 43, which acts to pull the support plate 34 towards the support bar 35.

The anatomical location at the contact area of support bar 35 is (as described above for the support bar 25 in connection with FIGS. 25-27) much more rigid relative to the heart muscle tissue at the anatomical location of the support plate 34. Therefore, the tension on the doubled tether 43 acts to apply traction to the segment of the relatively soft ventricle wall which is the base of the papillary muscles (similar to the situation described above in connection with FIG. 27). The directional traction of the papillary muscles acts to relieve tension from the mitral valve chordae tendinea and allow better coaptation of the leaflets and thereby improve the valve functioning.

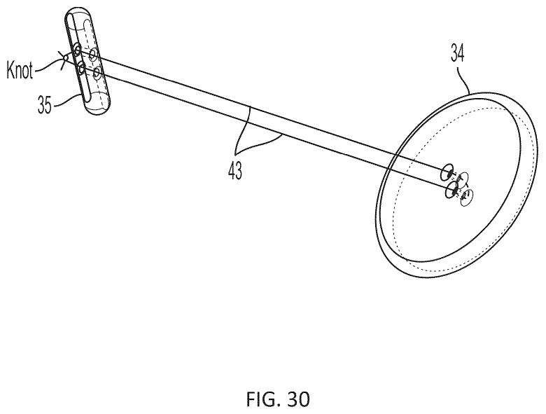

When the desired improvement in the valve functioning has been achieved, the doubled tether 43 is secured to the support bar 35 as depicted in FIG. 30 to maintain tension between the support plate 34 and the support bar 35 and preserve the improved hemodynamics. This can be accomplished e.g., using a knot or a clip (not shown). In some embodiments, the procedure is nearly complete after the knot has been tied or the clip has been installed. In these embodiments, the portion of the doubled tether 43 that is proximal with respect to the knot or clip is cut off and discarded, at which point the procedure is complete. In other embodiments, additional components can be installed after the knot/clip has been tied/installed. In these embodiments, the portion of the doubled tether 43 that is proximal with respect to the knot/clip is not cut off. Instead, that portion is used to connect to the additional components, in a manner that is analogous to the embodiments described below in connection with FIGS. 31-36.

Notably, in the embodiments described above in connection with FIGS. 28-30, the original tether 23 (which is used for advancing) and the doubled tether 43 (which is used for retracting) are distinct from each other, and the original tether 23 is used to thread the doubled tether 43 through both the first passageway and the second passageway.

FIGS. 31 AND 32 depict two views of another embodiment in which the secured position of support bar 25 described above serve as an Archimedean point to ameliorate functioning of the tricuspid valve by relieving tension forces on the chordae tendinea of that valve. This embodiment begins as described above in connection with FIGS. 20-27, but includes the additional steps described below.

After the position of the support bar 25 has been secured, a second needle is connected to a proximal extension of the tether 23 and advanced into the right ventricle, and a third puncture is created in the anterior wall of the right ventricle at a location between the subject's right-ventricular papillary muscles. This can be done by pushing the second needle through an outer wall of the right ventricle at a location between the subject's right-ventricular papillary muscles to create a third passageway (similar to how the second passageway was created, as described above). An extension of the tether 23 is exteriorized through the outer wall of the right ventricle, and a second support plate 26 is advanced over the tether in a proximal direction and though the ribs until the second support plate reaches an outer surface of the outer wall of the right ventricle adjacent to the third passageway. The second support plate is then pushed against the outer wall of the right ventricle, which will move the “roots” of the right-ventricular chordae to a position that will improve coaptation of the tricuspid valve leaflets. The tether is then fastened to the second support plate 26 e.g., using a suitable clip or knot while the tether is under tension. Note that in this example, a single tether 23 is depicted. The portion of this tether that runs between the support bar 25 and the first support plate 24 is referred to in this section as the first tether, and the portion of the tether that runs between the support bar 25 and the second support plate 26 is referred to in this section as the second tether. But in alternative embodiments, two separate tethers may be used instead of a single tether.

In a variation of the FIG. 31/32 embodiment, two separate tethers are used, with one tether running between the support bar 25 and the first support plate 24, and a separate second tether that runs between the support bar 25 and the second support plate 26. This variation begins as described above in connection with FIGS. 20-27, but includes the additional steps described below. A second needle is introduced into the right ventricle, with the second tether affixed to the second needle. The second needle is pushed through an anterior wall of the right ventricle at a location between the subject's right-ventricular papillary muscles to create a third passageway (similar to how the second passageway was created, as described above. The second needle is advanced in a distal direction until a distal portion of the second tether exits the heart. The second tether is advanced in a distal direction until a portion of the second tether exits the subject's body. The second support plate 26 is secured to the second tether e.g., in a manner similar to how the first support plate 24 was secured to the first tether 23. The second tether is then retracted and the second support plate 26 is moved in a proximal direction between the subject's ribs until it reaches an outer surface of the outer wall of the right ventricle adjacent to the third passageway. (This is similar to the way the first support plate 24 was moved in a proximal direction until it reached the outer wall of the left ventricle, as described above.) The second tether is then pulled in a proximal direction to create tension, and subsequently the second tether is secured to the support bar 25 under tension (e.g., using a knot or clip that is inserted via a suitable catheter).

Note that both of the variations of the FIG. 31/32 embodiment described above can be combined with the doubled tether embodiment described above in connection with FIGS. 28-30. When this combination is implemented, a doubled tether can be used to hold the second support plate against the outer surface of the outer wall of the right ventricle adjacent to the third passageway (instead of the single tether described in the two previous paragraphs). This can be accomplished e.g., using techniques similar to those described above in connection with FIGS. 28-30.

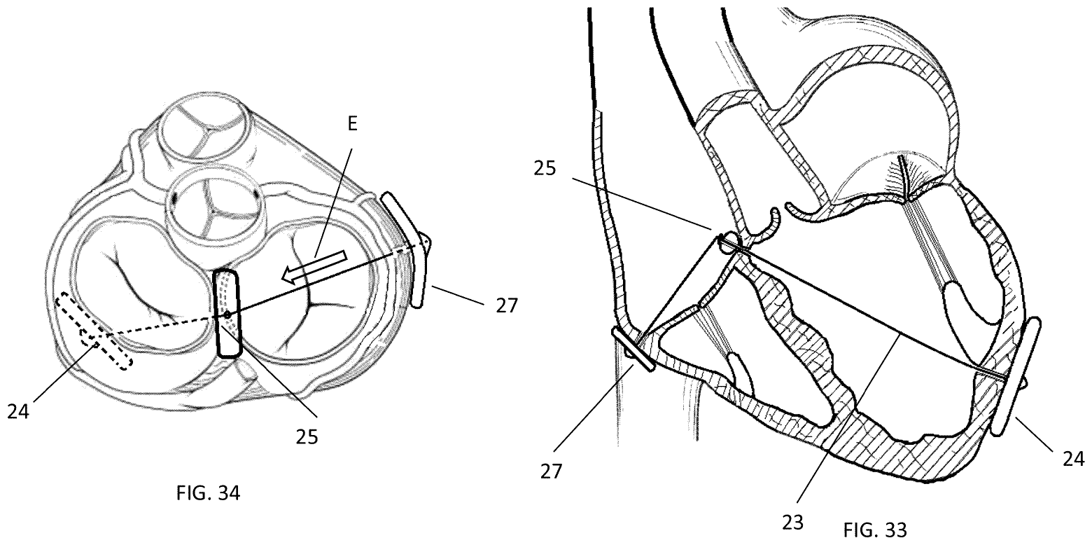

FIGS. 33 AND 34 depict two views of another embodiment in which the secured position of support bar 25 described above serves as an Archimedean point to deform the tricuspid valve annulus to improve the coaptation of that valve. This embodiment begins as described above in connection with FIGS. 20-27, but includes the additional steps described below.

After the position of the support bar 25 has been secured, a second needle is connected to a proximal extension of the tether 23 and advanced into the right atrium, and a third puncture is created through an anterior outer wall of the right atrium at a location above the tricuspid valve to create a third passageway. An extension of the tether 23 is exteriorized through the outer wall of the right atrium, and a curved support member 27 is advanced over the tether in a proximal direction and though the ribs until the curved support member reaches an outer surface of the outer wall of the right atrium adjacent to the third passageway. The curved support member 27 has a curvature that fits the outer wall of the right atrium. The curved support member 27 is then pushed against the outer wall of the right atrium, which will deform the tricuspid valve annulus and improve coaptation of the tricuspid valve leaflets. The tether is then fastened to the curved support member 27 e.g., using a suitable clip or knot at a position where the tether is under tension. Note that in this example, a single tether 23 is depicted. The portion of this tether that runs between the support bar 25 and the first support plate 24 is referred to in this section as the first tether, and the portion of the tether that runs between the support bar 25 and the second curved support member 27 is referred to in this section as the second tether. But in alternative embodiments, two separate tethers may be used instead of a single tether.

In a variation of the FIG. 33/34 embodiment, two separate tethers are used, with one tether running between the support bar 25 and the first support plate 24, and a separate second tether that runs between the support bar 25 and the curved support member 27. This variation begins as described above in connection with FIGS. 20-27, but includes the additional steps described below. A second needle is introduced into the right atrium, with a second tether affixed to the second needle. The second needle is pushed through an anterior outer wall of the right atrium at a location above the tricuspid valve to create a third passageway. The second needle is advanced in a distal direction until a distal portion of the second tether exits the heart. The second tether is then advanced in a distal direction until a portion of the second tether exits the subject's body. The curved support member 27 is secured to the second tether. The curved support member 27 has a curvature that fits the outer wall of the right atrium. The second tether is then retracted and the curved support member 27 is moved in a proximal direction between the subject's ribs until it reaches an outer surface of the outer wall of the right atrium adjacent to the third passageway. The second tether is then pulled in a proximal direction to create tension, and subsequently the second tether is secured to the support bar 25 under tension.

Note that both of the variations of the FIG. 33/34 embodiment described above can be combined with the doubled tether embodiment described above in connection with FIGS. 28-30. When this combination is implemented, a doubled tether can be used to hold the curved support member against the outer surface of the outer wall of the right atrium adjacent to the third passageway (instead of the single tether described in the two previous paragraphs). This can be accomplished e.g., using techniques similar to those described above in connection with FIGS. 28-30.

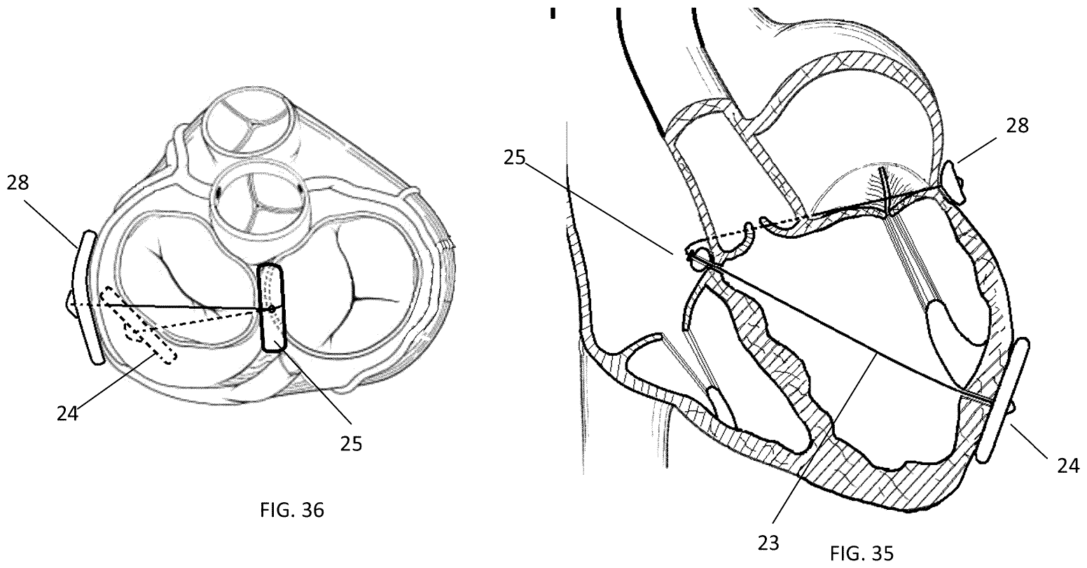

FIGS. 35 and 36 depict two views of another embodiment in which the secured position of support bar 25 described above serve as an Archimedean point to deform the mitral valve annulus to improve the coaptation of that valve. This embodiment begins as described above in connection with FIGS. 20-27, but includes the additional steps described below.

After the position of the support bar 25 has been secured, a second needle is connected to a proximal extension of the tether 23 and introduced into the right atrium and advanced through the heart until it enters the left atrium. The second needle is then used to create a third puncture through a posterior outer wall of the left atrium at a location above the mitral valve to create a third passageway. An extension of the tether 23 is exteriorized through the outer wall of the left atrium, and a curved support member 28 is advanced over the tether in a proximal direction and though the ribs until the curved support member reaches an outer surface of the outer wall of the left atrium adjacent to the third passageway. The curved support member 28 has a curvature that fits the outer wall of the left atrium. The curved support member 28 is then pushed against the outer wall of the left atrium, which will deform the mitral valve annulus and improve coaptation of the mitral valve leaflets. The tether is then fastened to the curved support member 28 e.g., using a suitable clip or knot while the tether is under tension. Note that in this example, a single tether 23 is depicted. The portion of this tether that runs between the support bar 25 and the first support plate 24 is referred to in this section as the first tether, and the portion of the tether that runs between the support bar 25 and the second curved support member 28 is referred to in this section as the second tether. But in alternative embodiments, two separate tethers may be used instead of a single tether.

In a variation of the FIG. 35/36 embodiment, two separate tethers are used, with one tether running between the support bar 25 and the first support plate 24, and a separate second tether that runs between the support bar 25 and the curved support member 28. This variation begins as described above in connection with FIGS. 20-27, but includes the additional steps described below. A second needle is introduced into the right atrium, with a second tether affixed to the second needle. The second needle is advanced through the heart until it enters the left atrium and is then pushed through a posterior outer wall of the left atrium at a location above the mitral valve to create a third passageway. The second needle is advanced in a distal direction until a distal portion of the second tether exits the heart. The second tether is then advanced in a distal direction until a portion of the second tether exits the subject's body. The curved support member 28 is secured to the second tether. The curved support member 28 has a curvature that fits the outer wall of the left atrium. The second tether is then retracted and the curved support member 28 is moved in a proximal direction between the subject's ribs until it reaches an outer surface of the outer wall of the left atrium adjacent to the third passageway. The second tether is then pulled in a proximal direction to create tension, and subsequently the second tether is secured to the support bar 25 under tension.

Note that both of the variations of the FIG. 35/36 embodiment described above can be combined with the doubled tether embodiment described above in connection with FIGS. 28-30. When this combination is implemented, a doubled tether can be used to hold the curved support member against the outer surface of the outer wall of the left atrium adjacent to the third passageway (instead of the single tether described in the two previous paragraphs). This can be accomplished e.g., using techniques similar to those described above in connection with FIGS. 28-30.

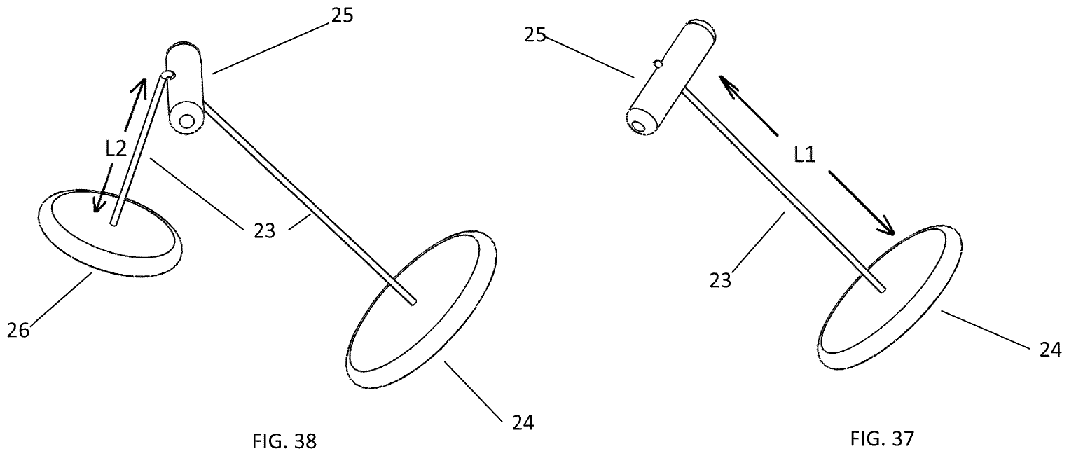

FIG. 37 depicts the components of the FIG. 27 embodiment, with labels for indicating corresponding dimensions. The values for the various dimensions that are suitable for small, medium, and large sized adults are provided below in table 1. All dimensions in table 1 are in millimeters.

TABLE 1

| | Small | Medium | Large |
|---|---|---|---|
| Component | subject | subject | subject |
| First support plate 24 diameter | 20-40 | 25-45 | 30-50 |
| Support bar 25 diameter | 3-8 | 3-8 | 3-8 |
| Support bar 25 length | 10-20 | 15-25 | 20-30 |
| Second support plate 26 diameter | 20-40 | 25-45 | 30-50 |
| Curved support member 27 radius R1 | 15-18 | 17-20 | 20-23 |
| Curved support member 27 height H1 | 8 | 8 | 8 |
| Alt. support bar 29 radius R2 | 15-18 | 17-20 | 20-23 |
| Alt. support bar 29 height H2 | 8 | 8 | 8 |
| Tether 23 length L1 | 60-85 | 65-90 | 70-95 |
| Tether 23 length L2 | 40-60 | 50-70 | 60-80 |
| Tether 23 length L3 | 25-40 | 30-45 | 35-50 |

FIG. 38 depicts the components of the FIG. 31 embodiment, with labels for indicating corresponding dimensions. The values for the various dimensions that are suitable for small, medium, and large sized adults are provided above in table 1.

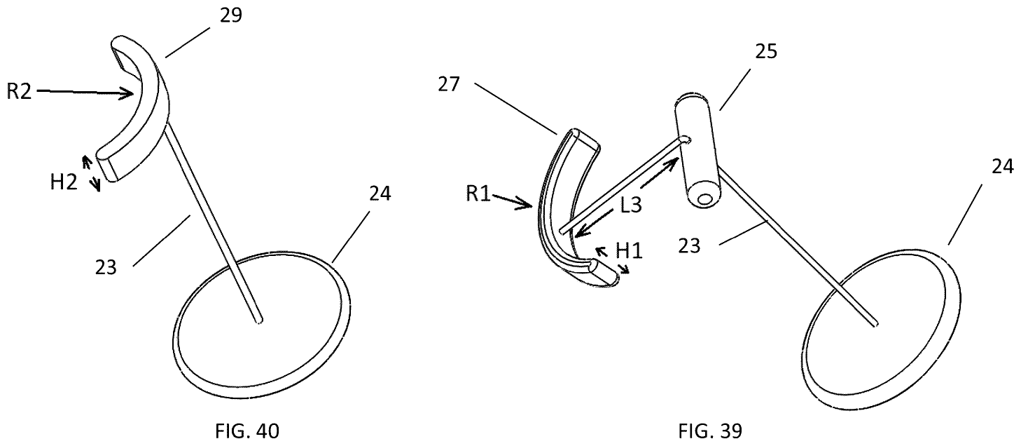

FIG. 39 depicts the components of the FIG. 33 embodiment, with labels for indicating corresponding dimensions. The values for the various dimensions that are suitable for small, medium, and large sized adults are provided above in table 1.

In FIGS. 24-38, the first support member is depicted as being a straight cylindrical support bar 25/35. And as explained above, the support bar 25/35 does not have to be cylindrical. Furthermore, the support bar need not be straight. FIG. 40 depicts an embodiment that is similar to the FIG. 27 embodiment, except that the straight support bar 25 from the FIG. 27 embodiment is replaced with a curved support bar 29. The labels in FIG. 40 indicate corresponding dimensions, and the values for the various dimensions that are suitable for small, medium, and large sized adults are provided above in table 1.

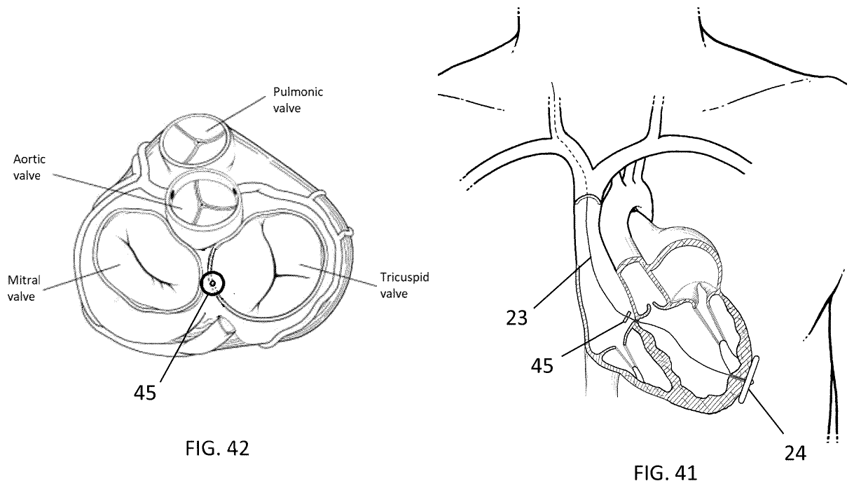

Further still, the first support member can be implemented using a member that is not bar-shaped, as described below in connection with FIGS. 41-42.

FIGS. 41-42 depict another embodiment for moving the posterior wall of the left ventricle so that it more closely resembles the healthy person's heart depicted in FIG. 18. Unlike the situation described above in connection with FIGS. 20-40, the first support member in this FIGS. 41-42 embodiment is not bar-shaped. Instead, the first support member in this embodiment is a non-elongated support member 45. Examples of suitable shapes for the non-elongated support member 45 include round (e.g., as depicted in FIGS. 41-42), square, hexagonal, octagonal, and other non-elongated shapes. The shape of the non-elongated support member 45 should be such that the non-elongated support member can be delivered via a catheter.

This embodiment begins in the same manner described above in connection with FIGS. 20-24, up to and including the point where the first support plate 24 reaches the outer surface of the outer wall of the left ventricle adjacent to the second passageway.

Referring now to FIGS. 41-42, a non-elongated support member 45 is then advanced over the tether 23 via the vascular system until it contacts the tissue at the site of the puncture between the right atrium and the left ventricle.

The first support plate 24 and the non-elongated support member 45 can be made from a suitable molded or machined biocompatible plastic (including but not limited to PEEK, PC, PSU, and/or ABS). And the tether 23 can be a suitable braided plastic with a high tensile strength (including but not limited to nylon and/or ultra-high-molecular-weight polyethylene).

The location of the non-elongated support member 45 just before it has reached this position is depicted in FIG. 41 (a cross section of the heart chambers' view). And the location of the non-elongated support member 45 after it has reached its final position is depicted in FIG. 42 (a cross section of the heart valves plane). More specifically, in its final position, the non-elongated support member 45 should be positioned within the right atrium in contact with a septal portion of the tricuspid annulus, and adjacent to the first passageway.

In some preferred embodiments, the non-elongated support member 45 has a through-hole 45T, and the tether 23 is threaded through this through hole 45T before the non-elongated support member 45 is delivered into the right atrium by advancing it over the tether 23. In the illustrated embodiment, the through hole 45T is in the center of the non-elongated support member 45. But in alternative embodiments, the through hole 45T could be offset from the center e.g., by up to 25% of the non-elongated support member's length. A suitable length for the non-elongated support member 45 is 3-8 mm for small adults, 7-12 mm for medium-sized adults, and 11-16 mm for large adults. The dimensions of all the other components in this FIGS. 41-42 embodiment are the same as in the embodiments described above in connection with FIGS. 20-40.

A pull force in the proximal direction is then applied on the tether 23 while maintaining the position of the non-elongated support member 45 in contact with the tissue. The pull force may be applied by pulling a portion of the tether 23 that remains outside of the subject's body in a proximal direction. And the position of the non-elongated support member 45 may be maintained by using a catheter-based tool to push the non-elongated support member 45 against the tricuspid annulus. This creates tension in the tether 23, which acts to pull the first support plate 24 towards the non-elongated support member 45.

As described above in connection with FIG. 27, the anatomical location at the contact area of the non-elongated support member 45 is a central part of the fibrotic skeleton of the heart, which is much more rigid relative to the heart muscle tissue at the anatomical location of the first support plate 24. The tension on tether 23 will therefore pull the base of the papillary muscles close to the mitral valve, which will relieve tension from the mitral valve chordae tendinea. This will allow better coaptation of the leaflets and thereby improve the valve functioning.

When the desired improvement in the valve functioning has been achieved, tether 23 is secured to the non-elongated support member 45 e.g., using a knot or a clip (not shown) to maintain tension between the first support plate 24 and the non-elongated support member 45 and preserve the improved hemodynamics. In some embodiments, the procedure is nearly complete after the knot has been tied or the clip has been installed. In these embodiments, the portion of the tether 23 that is proximal with respect to the knot or clip is cut off and discarded, at which point the procedure is complete. In other embodiments, additional components can be installed after the knot/clip has been tied/installed. These embodiments are similar to the embodiments described above in connection with FIGS. 31-36, except that a non-elongated support member 45 is used instead of the support bar 25.

Note that the FIG. 41-42 embodiment is described in the context of a single tether 23 that is (a) initially advanced from the right atrium into the left ventricle, (b) subsequently advanced from the left ventricle until it exits the subject's heart, (c) subsequently advanced until it exits the subject's body, and (d) subsequently retracted until the first support plate reaches an outer surface of the outer wall of the left ventricle adjacent to the second passageway. But in alternative embodiments, distinct tethers can be used for the advancing function and the retracting function, analogous to the approach described above in connection with FIGS. 28-30, except that a non-elongated support member 45 is used instead of the support bar 25.

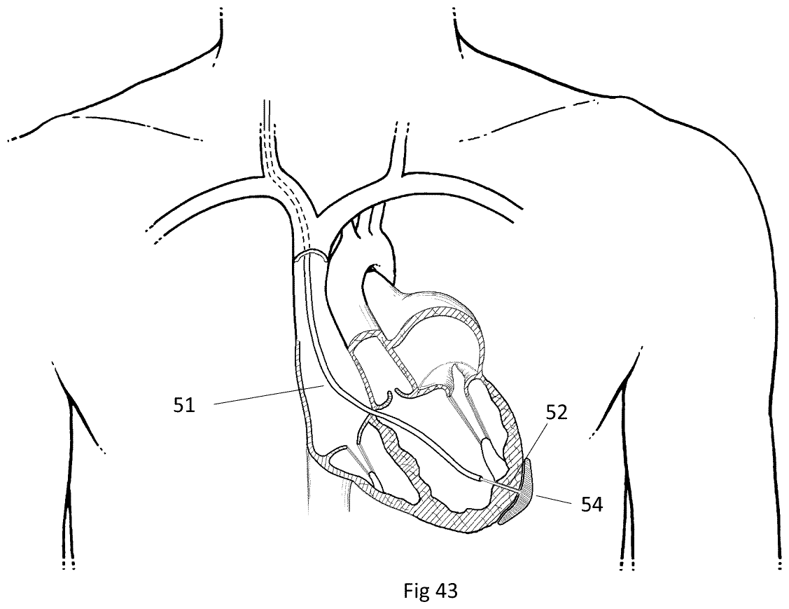

FIG. 43 depicts an alternative embodiment for positioning a support plate against an outer surface of a posterior wall of the heart near the two left-ventricular papillary muscles, and positioning a first support member (e.g., a support bar or a non-elongated support member) within the heart's right atrium, against a surface of the heart's tricuspid annulus that is adjacent to a posterior wall of the right atrium, with a tether under tension connecting the support plate and the first support member (e.g., the support bar or the non-elongated support member).

This embodiment begins as described above in connection with FIGS. 20-22, up to the point where the catheter is advanced through the first passageway into the left ventricle. A needle (similar to the needle 22 described above in connection with FIGS. 20-22) is advanced through the catheter 51 to a location in the outer wall of the LV that is near at least one of the left-ventricular papillary muscles, and more preferably between the two left-ventricular papillary muscles. The needle is then pushed through the outer wall of the LV to puncture the outer wall of the LV (again similar to the needle 22 described above in connection with FIGS. 20-22). The puncture in the outer wall of the LV serves as a second passageway.

A second, smaller catheter 52 incorporating a thin wall bag at the distal end is tracked through catheter 51 and over the needle through the second passageway, and the needle is pulled back and removed from the patient's body. The thin wall bag is then expanded and spread radially into a flat mushroom shape outside the ventricle wall as depicted in FIG. 43. The thin wall bag is then filled with solidifying polymer, which upon solidification will become a rigid structural plate 54 that is attached to a tether. Details on how to form a rigid structural plate at this location with an attached tether are described in U.S. Pat. No. 10,299,928, which is incorporated herein by reference in its entirety.

After the rigid structural plate 54 has been formed and fully cured, a support bar (or a first support member that is non-elongated) is advanced over the tether that is attached to the rigid structural plate 54. When the first support member is a support bar, the installation of this support bar (and the nature of the support bar itself) can be similar to the support bar 25 described above in connection with FIGS. 25-27. In alternative embodiments, the first support member can be similar to any of the non-elongated support members described above.

Note that in the embodiments described above, the various passageways (e.g., the first passageway, the second passageway, etc.) are created using a conventional needle that is pushed through a respective portion of tissue in the subject's body. As a result, the creation of the various passageways and the pushing of the needle through those passageways occur simultaneously. But in alternative embodiments, any of these passageways could be created first (e.g., using an RF needle), and a needle could then be pushed through the passage.

Finally, it is important to note that the usage of the identifiers (a), (b), (c), (d), etc. in the claims below does not imply a particular sequence in time for the corresponding steps. For while it is certainly possible that step (a) will precede step (b) in time, different sequencings of those steps are also possible, except in cases where a particular sequencing is inconsistent with the internal language of the various steps or with other language in the claims. For example, a step labeled (b) could precede a step labeled (a) in time. It is also possible for two or more steps to occur simultaneously or to overlap to an extent, except in cases where simultaneity or overlapping would be inconsistent with the internal language of the various steps or with other language in the claims.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of ameliorating mitral regurgitation in a heart within a subject's body, the heart having a right atrium, a right ventricle, a left ventricle, two left-ventricular papillary muscles, an interventricular septum, a tricuspid valve, and a tricuspid annulus, the method comprising:
creating a first passageway between the right atrium and the left ventricle at a septal portion of the tricuspid annulus that is directly above the interventricular septum;
creating a second passageway through an outer wall of the left ventricle near at least one of the left-ventricular papillary muscles;
positioning a first support member within the right atrium in contact with a septal portion of the tricuspid annulus;
positioning a first support plate against the outer wall of the left ventricle adjacent to the second passageway; and
pulling the first support plate towards the first support member using a first tether under tension, wherein the first tether runs between the first support plate and the first support member and passes through both the first passageway and the second passageway, and wherein a first end of the first tether is secured to the first support plate.

2. The method of claim 1, wherein the second passageway is located between the two left-ventricular papillary muscles.

3. The method of claim 1, wherein a second end of the first tether is secured to the first support member.

4. The method of claim 1, wherein the first support member is non-elongated.

5. The method of claim 4, wherein the first support plate has an area of 3-13 cm²,
wherein the first support member has a length of 3-8 mm, and
wherein the first tether has a length of 60-85 mm.

6. The method of claim 4, wherein the first support plate has an area of 4-16 cm,
wherein the first support member has a length of 7-12 mm, and
wherein the first tether has a length of 65-90 mm.

7. The method of claim 4, wherein the first support plate has an area of 7-20 cm²,
wherein the first support member has a length of 11-16 mm, and
wherein the first tether has a length of 70-95 mm.

8. The method of claim 1, wherein the first support member is bar-shaped.

9. The method of claim 8, wherein the first support plate has an area of 3-13 cm², and
wherein the first support member has a length of 10-20 mm and a width of 3-8 mm.

10. The method of claim 8, wherein the first support plate has an area of 4-16 cm², and
wherein the first support member has a length of 15-25 mm and a width of 3-8 mm.

11. The method of claim 8, wherein the first support plate has an area of 7-20 cm², and
wherein the first support member has a length of 20-30 mm and a width of 3-8 mm.

12. The method of claim 1, wherein the positioning of the first support plate against the outer wall of the left ventricle comprises:
positioning a polymer bag outside the outer wall of the left ventricle adjacent to the second passageway;
injecting a liquid material into the polymer bag; and
allowing the liquid material to solidify,
wherein the solidification of the liquid material forms the first support plate.

13. The method of claim 1, further comprising:
creating a third passageway through an outer wall of the right ventricle near at least one right-ventricular papillary muscle;
positioning a second support plate against an outer wall of the right ventricle adjacent to the third passageway; and
pulling the second support plate towards the first support member using a second tether under tension, wherein the second tether runs between the second support plate and the first support member, and wherein a first end of the second tether is secured to the second support plate.

14. The method of claim 1, further comprising:
positioning a second support member against an outer wall of the heart at a second location adjacent to an anterior leaflet of the tricuspid valve; and
pulling the second support member towards the first support member using a second tether under tension, wherein the second tether runs between the second support member and the first support member, and wherein a first end of the second tether is secured to the second support member.

15. The method of claim 1, further comprising:
positioning a second support member against an outer wall of the heart at a second location adjacent to a posterior leaflet of a mitral valve; and
pulling the second support member towards the first support member using a second tether under tension, wherein the second tether runs between the second support member and the first support member, and wherein a first end of the second tether is secured to the second support member.

16. A method of ameliorating mitral regurgitation in a heart within a subject's body, the heart having a right atrium, a left atrium, a right ventricle, a left ventricle, two left-ventricular papillary muscles, an interventricular septum, a tricuspid valve, a mitral valve, and a cardiac skeleton, the method comprising:
(a) introducing a first needle into the right atrium, with a first advancing tether affixed to the first needle;
(b) creating a first passageway between the right atrium and the left ventricle through the cardiac skeleton at a location that is directly above the interventricular septum and pushing the first needle through the first passageway into the left ventricle;
(c) creating a second passageway through an outer wall of the left ventricle at a location between the left-ventricular papillary muscles and pushing the first needle through the second passageway;
(d) advancing the first needle in a distal direction until a distal portion of the first advancing tether exits the heart;
(e) advancing the first advancing tether in a distal direction until a portion of the first advancing tether exits the subject's body;
(f) securing a first support plate to a first retracting tether that is threaded through both the first passageway and the second passageway;
(g) retracting the first retracting tether and moving the first support plate in a proximal direction between the subject's ribs until the first support plate reaches an outer surface of the outer wall of the left ventricle adjacent to the second passageway;
positioning a first support member within the right atrium adjacent to the first passageway; and
after steps (a) through (g), pulling the first retracting tether in a proximal direction while the first support member is positioned adjacent to the first passageway, and subsequently securing the first retracting tether to the first support member under tension.

17. The method of claim 16, wherein a single tether serves as both the first advancing tether and the first retracting tether.

18. The method of claim 16, wherein the first advancing tether and the first retracting tether are distinct from each other, and wherein the first advancing tether is used to thread the first retracting tether through both the first passageway and the second passageway.

19. The method of claim 16, wherein the first support member is non-elongated.

20. The method of claim 16, wherein the first support member is bar-shaped.

21. The method of claim 16, further comprising:
introducing a second needle into the right ventricle, with a second tether affixed to the second needle;
creating a third passageway through an outer wall of the right ventricle at a location between the subject's right-ventricular papillary muscles and pushing the second needle through the third passageway;
advancing the second needle in a distal direction until a distal portion of the second tether exits the heart;
advancing the second tether in a distal direction until a portion of the second tether exits the subject's body;
advancing a second support plate over the second tether in a proximal direction until the second support plate reaches an outer surface of the outer wall of the right ventricle adjacent to the third passageway;
pushing the second support plate against the outer wall of the right ventricle; and
securing one portion of the second tether to the second support plate and securing another portion of the second tether to the first support member so that the second tether is under tension.

22. The method of claim 16, further comprising:
introducing a second needle into the right ventricle, with a second advancing tether affixed to the second needle;
creating a third passageway through an outer wall of the right ventricle at a location between the subject's right-ventricular papillary muscles and pushing the second needle through the third passageway;
advancing the second needle in a distal direction until a distal portion of the second advancing tether exits the heart;
advancing the second advancing tether in a distal direction until a portion of the second advancing tether exits the subject's body;
securing a second support plate to a second retracting tether that is threaded through the third passageway;
retracting the second retracting tether and moving the second support plate in a proximal direction between the subject's ribs until the second support plate reaches an outer surface of the outer wall of the right ventricle adjacent to the third passageway; and
pulling the second retracting tether in a proximal direction, and subsequently securing the second retracting tether to the first support member under tension.

23. The method of claim 16, further comprising:
introducing a second needle into the right atrium, with a second tether affixed to the second needle;
creating a third passageway through an anterior outer wall of the right atrium at a location above the tricuspid valve and pushing the second needle through the third passageway;
advancing the second needle in a distal direction until a distal portion of the second tether exits the heart;
advancing the second tether in a distal direction until a portion of the second tether exits the subject's body;
advancing a curved support member over the second tether in a proximal direction until the curved support member reaches an outer surface of the outer wall of the right atrium adjacent to the third passageway, wherein the curved support member has a curvature that fits the outer wall of the right atrium;
pushing the curved support member against the outer wall of the right atrium; and
securing one portion of the second tether to the curved support member and securing another portion of the second tether to the first support member so that the second tether is under tension.

24. The method of claim 16, further comprising:
introducing a second needle into the right atrium, with a second advancing tether affixed to the second needle;
creating a third passageway through an anterior outer wall of the right atrium at a location above the tricuspid valve and pushing the second needle through the third passageway;
advancing the second needle in a distal direction until a distal portion of the second advancing tether exits the heart;
advancing the second advancing tether in a distal direction until a portion of the second advancing tether exits the subject's body;
securing a curved support member to a second retracting tether that is threaded through the third passageway, wherein the curved support member has a curvature that fits the outer wall of the right atrium;
retracting the second retracting tether and moving the curved support member in a proximal direction between the subject's ribs until the curved support member reaches an outer surface of the outer wall of the right atrium adjacent to the third passageway; and
pulling the second retracting tether in a proximal direction, and subsequently securing the second tether to the first support member under tension.

25. The method of claim 16, further comprising:
introducing a second needle into the right atrium, with a second tether affixed to the second needle;
advancing the second needle through the heart until the second needle enters the left atrium;
creating a third passageway through a posterior outer wall of the left atrium at a location above the mitral valve and pushing the second needle through the third passageway;
advancing the second needle in a distal direction until a distal portion of the second tether exits the heart;
advancing the second tether in a distal direction until a portion of the second tether exits the subject's body;
advancing a curved support member over the second tether in a proximal direction until the curved support member reaches an outer surface of the outer wall of the left atrium adjacent to the third passageway, wherein the curved support member has a curvature that fits the outer wall of the left atrium;
pushing the curved support member against the outer wall of the left atrium; and
securing one portion of the second tether to the curved support member and securing another portion of the second tether to the first support member so that the second tether is under tension.

26. The method of claim 16, further comprising:
introducing a second needle into the right atrium, with a second advancing tether affixed to the second needle;
advancing the second needle through the heart until the second needle enters the left atrium;
creating a third passageway through a posterior outer wall of the left atrium at a location above the mitral valve and pushing the second needle through the third passageway;
advancing the second needle in a distal direction until a distal portion of the second advancing tether exits the heart;
advancing the second advancing tether in a distal direction until a portion of the second advancing tether exits the subject's body;
securing a curved support member to a second retracting tether that is threaded through the third passageway, wherein the curved support member has a curvature that fits the outer wall of the left atrium;
retracting the second retracting tether and moving the curved support member in a proximal direction between the subject's ribs until the curved support member reaches an outer surface of the outer wall of the left atrium adjacent to the third passageway; and
pulling the second retracting tether in a proximal direction, and subsequently securing the second tether to the first support member under tension.

27. An apparatus for ameliorating mitral regurgitation in a heart within a subject's body, the heart having two left-ventricular papillary muscles, each of which has a respective center, the apparatus comprising:
a support plate configured for being positioned against an outer surface of a posterior wall of the heart, at a location that is within 3 cm of a midpoint between the centers of the two left-ventricular papillary muscles,
a first support member configured for being positioned within the heart's right atrium, against a surface of the heart's tricuspid annulus that is adjacent to a posterior wall of the right atrium; and
a tether that runs between the support plate and the first support member, wherein the tether is under tension so that the support plate is pulled towards the first support member.

28. The apparatus of claim 27, wherein the support plate is configured to be positioned against the outer surface of the posterior wall of the heart, at a location that is within 2 cm of the midpoint between the centers of the two left-ventricular papillary muscles.

29. The apparatus of claim 27, wherein the first support member is configured to be positioned adjacent to a first passageway that runs between the subject's right atrium and the subject's left ventricle,
wherein the support plate is configured to be positioned adjacent to a second passageway that runs through an outer wall of a subject's left ventricle,
wherein the tether is configured to pass through both the first passageway and the second passageway, and
wherein a first portion of the tether is secured to the first support member and a second portion of the tether is secured to the support plate.

30. The apparatus of claim 27, wherein the first support member is non-elongated.

31. The apparatus of claim 30, wherein the support plate has an area of 3-13 cm²,
wherein the first support member has a length of 3-8 mm, and
wherein the tether has a length of 60-85 mm.

32. The apparatus of claim 30, wherein the support plate has an area of 4-16 cm,
wherein the first support member has a length of 7-12 mm, and
wherein the tether has a length of 65-90 mm.

33. The apparatus of claim 30, wherein the support plate has an area of 7-20 cm²,
wherein the first support member has a length of 11-16 mm, and
wherein the tether has a length of 70-95 mm.

34. The apparatus of claim 27, wherein the first support member is bar-shaped.

35. The apparatus of claim 34, wherein the support plate has an area of 3-13 cm²,
wherein the first support member has a length of 10-20 mm and a width of 3-8 mm, and
wherein the tether has a length of 60-85 mm.

36. The apparatus of claim 34, wherein the support plate has an area of 4-16 cm²,
wherein the first support member has a length of 15-25 mm and a width of 3-8 mm, and
wherein the tether has a length of 65-90 mm.

37. The apparatus of claim 34, wherein the support plate has an area of 7-20 cm²,
wherein the first support member has a length of 20-30 mm and a width of 3-8 mm, and
wherein the tether has a length of 70-95 mm.
